(19) United States
(12) Patent Application Publication
(10) Pub. No.: US 2023/0190783 A1
(43) Pub. Date: Jun. 22, 2023

(54) DELIVERY OF LOW VISCOSITY FORMULATIONS

(71) Applicant: Jazz Pharmaceuticals Ireland Limited, Dublin (IE)

(72) Inventors: Qi WANG, Pennington, NJ (US); Sekhar R. KANAPURAM, Thousand Oaks, CA (US); James C. LEAMON, Warminster, PA (US); Mariana DIMITROVA, Wallingford, PA (US)

(21) Appl. No.: 17/802,795

(22) PCT Filed: Feb. 26, 2021
(86) PCT No.: PCT/US2021/019964

§ 371 Date: Aug. 26, 2022

Related U.S. Application Data
(60) Provisional application No. 62/983,023, filed on Feb. 28, 2020.

(51) Int. Cl. A61K 31/7105; A61K 47/18; A61K 9/00; A61M 5/142; A61M 5/145; A61M 5/32

(52) U.S. Cl. CPC A61K 31/7105; A61K 47/183; A61K 9/0009; A61M 5/14248; A61M 5/1452; A61M 5/3286

(57) ABSTRACT

Low-viscosity nucleic acid compositions that can be administered by oral or multiple parenteral routes may allow for less frequent dosing than nucleic acid products currently on the market. In particular, low-viscosity defibrotide formulations for subcutaneous, intramuscular, intradermal, and intraperitoneal administration are more convenient to the patient and/or are administered outside of the hospital setting. Formulations of the invention may be used for the treatment of numerous conditions including for example, treatment of peripheral arteriopathies, treatment of acute renal insufficiency, treatment of acute myocardial ischemia, and treatment and prevention of sinusoidal obstruction syndrome or VOD.

### 1. CROSS-REFERENCE TO RELATED APPLICATIONS

This application is a National Phase of International Application No. PCT/US2021/019964 filed Feb. 26, 2021, which claims priority to U.S. Provisional Application No. 62/983,023 filed Feb. 28, 2020. The contents of each of the aforementioned patent applications are incorporated herein by reference in their entireties.

### 2. BACKGROUND OF THE INVENTION

Defibrotide, a nucleic acid salt, is a complex mixture of random sequence, predominantly single-stranded polydeoxyribonucleotides derived from animal mucosal DNA. It has protective effects on vascular endothelial cells, particularly those of small vessels and has antithrombotic, anti-inflammatory and antiischemic properties.

The sodium salt of defibrotide is commercially sold as Defitelio® (Gentium S.r.L., Villa Guardia, Italy) and is currently approved for the treatment of adult and pediatric patients with hepatic veno-occlusive disease (VOD), also known as sinusoidal obstruction syndrome (SOS), with renal or pulmonary dysfunction following hematopoietic stem-cell transplantation (HSCT). It can be administered to patients by 2-hour intravenous infusions every 6 hours for a minimum of 21 days. However, this administration route, frequency and large volumes of the infusion regimen is generally not compatible in an outpatient dosing for disease indications for which defibrotide may be shown to be therapeutic. Therefore, it would be beneficial to administer defibrotide in a way that is more convenient to the patient to allow dosing in an outpatient setting, allow patients to self-administer at home via a compatible administration device, or reduce dosing duration and liquid volume in a hospital setting. Thus there is a need for new avenues of administration of defibrotide formulations, and new formulations of defibrotide which would permit new and more patient convenient dosing regimens for administration of pharmaceutically effective doses at home.

### 3. SUMMARY OF THE INVENTION

The present disclosure provides a broad range of nucleic acids and their salts, including defibrotide. In some aspects, the present disclosure provides high concentration formulations of these molecules while keeping the viscosity and osmolality at physiologically relevant levels. Subcutaneous administration of these formulations, including high concentration formulations, offers numerous benefits to the patient, including for example, the ability to be administered by the patient outside of a hospital setting. The advantages of self-administration and/or administration by other than the IV route are felt by the patient and their families as well as by the hospital. The amount of time and resources that the hospital needs to treat and monitor these patients are significantly reduced which provides a reduced economic burden on both the hospital and the patient. The formulations provided herein are specifically related to defibrotide; however, it is understood that the invention applies to a broad range of nucleotide products, for example, single and double-stranded DNA or RNA products, such as DNA and RNA vaccines.

Provided herein are nucleic acid compositions for therapeutic administration which may be administered by nasal, oral, intravenous, subcutaneous, or multiple parenteral routes and which may improve the quality of life for patients by less frequent and/or shorter duration of dosing than similar nucleic acid products currently on the market or the ability to administer via a device (e.g. a wearable device) as an out-patient. In some embodiments, the nucleic acid composition is a defibrotide formulation such as Defitelio® that is administered by routes including intravenous, subcutaneous, intramuscular, intradermal, intraocular, buccal, oral, inhaled, intranasal and/or intraperitoneal routes. In some embodiments, the nucleic acid composition is a are low-viscosity, high concentration nucleic acid formulation that can be administered by routes including intravenous, subcutaneous, intramuscular, intradermal, intraocular, buccal, oral, inhaled, intranasal and/or intraperitoneal routes. In certain embodiments, low-viscosity nucleic acid formulations are self-administered and/or administrated in an out-patient basis. In specific embodiments, the nucleic acid is defibrotide.

Formulations of the invention may be used for the treatment and/or prevention of numerous conditions including, for example, Hematopoietic Stem Cell Transplantation (HSCT) related complications such as sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome. Other conditions including, for example, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury in solid organ transplantation, Acute Respiratory Distress Syndrome (ARDS), Sickle Cell Vaso-occlusive Crisis (VOC) Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

In some aspects, the present disclosure provides low-viscosity pharmaceutical formulations comprising a nucleic acid at a concentration of at least 50 mg/mL. In some embodiments, the nucleic acid concentration is between about 80 mg/mL and about 400 mg/mL. In some embodiments, the viscosity of the formulation is: a) less than about 70 cP; b) between about 5 cP and 65 cP; or c) between about 10 cP and about 65 cP. In some embodiments, the viscosity is measured: a) at room temperature; b) between about 15° C. and about 35° C.; or c) between about 21° C. and about 23° C. In some embodiments, the low-viscosity formulation is Defitelio®.

In some embodiments, the low-viscosity pharmaceutical formulation further comprises a viscosity reducer. In some embodiments, the viscosity reducer is glycylglycine, glycine, sodium citrate, or benzyl alcohol. In some embodiments, the low-viscosity pharmaceutical formulation comprises benzyl alcohol at 0.25% to 2.0%. In some embodiments, the low-viscosity pharmaceutical formulation is citrate-free. In some embodiments, the viscosity reducer concentration is a) between about 5 mM and about 100 mM; b) between about 5 mM and 60 mM; or c) between about 10 mM and about 40 mM

In some embodiments, the low-viscosity pharmaceutical formulation has an osmolality of a) between about 240 mOsm/kg and about 1000 mOsm/kg; or b) between about 300 mOsm/kg and about 600 mOsm/kg.

In some embodiments, the nucleic acid in the low-viscosity pharmaceutical formulation comprises polynucleotide or oligonucleotides of ribonucleic acid or deoxyribonucleic acid. In some embodiments, the molecular weight of the nucleic acid is a) between about 5,000 to about 50,000 daltons; b) between about 13,000 to about 30,000 daltons; or c) between about 16,000 to about 20,000 daltons.

In some embodiments, the nucleic acid comprises polydisperse, random sequences. In some embodiments, the nucleic acid is present as predominantly single-stranded poly deoxyribonucleotides.

In some embodiments, the low-viscosity pharmaceutical formulation comprises single-stranded polydeoxyribonucleotides that are random sequences that correspond to the following formula:

P1-5,(dAp)12-24,(dGp)10-20,(dTp)13-26,(dCp)10-20 [symbol]
wherein: P=phosphoric radical
   dAp=deoxyadenylic monomer
   dGp=deoxyguanylic monomer
   dTp=deoxythymidylic monomer
   dCp=deoxycytidylic monomer

In some embodiments, the low-viscosity pharmaceutical formulation comprises a buffer or excipient selected from: acetate, histidine, phosphate, citrate, succinate, tartrate and maleate; a salt (e.g. sodium chloride, magnesium chloride and calcium chloride); and/or an amino acid (e.g. glycine, arginine, and proline or combinations thereof). In some embodiments, the low-viscosity pharmaceutical formulation comprises a buffer or excipient selected from: sodium citrate, sodium succinate, histidine (“HIS”), TRIS buffer, HEPES buffer, sodium acetate, sodium phosphate, lidocaine, succinic acid, acetic acid, phosphoric acid, tartaric acid, lidocaine, benzyl alcohol, sodium chloride, magnesium chloride, calcium chloride salts, amino acids (e.g. glycine, arginine, and proline or combinations thereof), cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, Solutol HS 15, hydroxypropyl betacyclodextrin, and/or polysorbate-80. In some embodiments, the low-viscosity pharmaceutical formulation is citrate-free. In some embodiments, the low-viscosity formulation comprises a buffer or excipient so that the nucleic acid is in the form of an alkali metal salt. In some embodiments, the buffer or excipient includes a sodium salt. In some embodiments, the buffer or excipient is sodium citrate, sodium succinate, sodium chloride, or a mixture thereof. In some embodiments, the buffer or excipient is sodium citrate, sodium succinate, sodium chloride, or a mixture thereof, at a concentration of less than about 80 mM sodium salt.

In some embodiments, the buffer or excipient is sodium citrate at a concentration of less than 34 mM, or between 20-34 mM. In some embodiments, the low-viscosity formulation comprises an agent that aides administration. In some embodiments, the low-viscosity formulation comprises an agent that aides subcutaneous administration. In some embodiments, the agent that aides subcutaneous administration is an enzyme. In some embodiments, the enzyme degrades hyaluronan (HA) in the subcutaneous space (e.g. HALOZYME®; a recombinant human hyaluronidase PH20 enzyme, rHuPH20, which locally degrades hyaluronan (HA) in the subcutaneous (SC) space; this allows for increased dispersion and absorption of co-administered therapies.)

In some aspects, the present disclosure provides low-viscosity pharmaceutical formulations comprising between 50 mg/mL to about 400 mg/mL of a composition comprising over 70% single-stranded, polydisperse polydeoxyribonucleotides, wherein each polydeoxribonucleotide comprises between 45 and 65 bases and has a mean molecular weight between 13 kDa and 20 kDa, and a viscosity reducer at a concentration of between about 5 mM and about 350 mM.

In some embodiments, the low viscosity defibrotide formulation comprises about 80 mg/mL of defibrotide and is formulated for subcutaneous or intravenous delivery to a patient. In some embodiments, the formulation is administered subcutaneously or intravenously delivered to a patient via a device.

In some embodiments, the low viscosity defibrotide formulation comprises about 50-200 mg/mL of defibrotide and about 10-350 mM viscosity reducer, and is formulated for subcutaneous delivery to a patient. In some embodiments, the low viscosity defibrotide formulation further comprises between about 10 mM to about 34 mM sodium citrate. In some embodiments, the low viscosity defibrotide formulation comprises about 180 mg/ml of defibrotide, about 10-350 mM viscosity reducer, and about 10-25 mM sodium citrate. In some embodiments, the low viscosity defibrotide formulation comprises about 120-200 mg/mL of defibrotide, about 10-350 mM viscosity reducer, and about 10-25 mM sodium citrate, wherein the formulation is formulated for subcutaneous or intravenous delivery to a patient. In some embodiments, the viscosity reducer is glycylglycine, glycine, sodium citrate, benzyl alcohol or a hyaluronidase (e.g. PH20).

In some embodiments, an agent which improves processing, such as a wetting agent or dispersing agent, is included in low viscosity defibrotide formulations of the invention. In specific embodiments, hyaluronidase is co-administered or co-formulated with a low viscosity defibrotide formulation of the invention.

In some aspects, the present disclosure provides low-viscosity pharmaceutical formulations comprising between 80 mg/mL to about 250 mg/mL of a nucleic acid composition comprising a nucleic acid over 70% single-stranded, polydisperse polydeoxyribonucleotides, wherein each polydeoxribonucleotide comprises between 45 and 65 bases and has a mean molecular weight between 13 kDa and 20 kDa, and a viscosity reducer at a concentration of between about 5 mM and about 60 mM, wherein the formulation has a viscosity between about 5 and about 70 cP when measured at between 15° C. and 25° C., and an osmolality between about 300 mOsm/kg and 600 mOsm/kg, and wherein the formulation is formulated for oral or parenteral administration to a patient.

In some aspects, the present disclosure provides low-viscosity pharmaceutical formulations comprising between 100 mg/mL to about 400 mg/mL of defibrotide, and a viscosity reducer at a concentration of between about 5 mM and about 350 mM, wherein the formulation has a viscosity between about 5 and about 70 cP when measured at between 15° C. and 25° C., and an osmolality between about 240 mOsm/kg and 1000 mOsm/kg, and wherein the formulation is formulated for oral or parenteral administration to a patient.

In some embodiments, the viscosity in the low-viscosity pharmaceutical formulation decreases over time. In some embodiments, the viscosity decreases during storage. In some embodiments, the viscosity decreases under increasing shear, agitation, and/or pressure. In some embodiments, the shear increases during administration of the pharmaceutical formulation. In some embodiments, the shear increases during administration of the pharmaceutical formulation via a needle or device.

In some embodiments, the low-viscosity pharmaceutical formulation is formulated for subcutaneous, intramuscular, intradermal, intraocular, buccal, oral, inhaled, intranasal, intravenous, or intraperitoneal administration. In some embodiments, the formulation demonstrates extended systemic half-life compared to a formulation delivered via intravenous administration or as a bolus. In some embodiments, the subcutaneously-delivered formulation exhibits lower peak-to-trough ratios of plasma concentrations compared to a formulation delivered via intravenous administration. In some embodiments, the subcutaneously-delivered formulation exhibits improves efficacy and/or an improved safety profile compared to a formulation delivered via intravenous administration.

In some embodiments, the low-viscosity pharmaceutical formulation isotonic, hypertonic or thixotropic.

In some embodiments, the low-viscosity pharmaceutical formulation may be self-administered by a patient.

In some aspects, the present disclosure provides a device for subcutaneous administration of low-viscosity formulations comprising a nucleic acid at a concentration of at least 50 mg/mL.

In some aspects, the present disclosure provides a device for out-patient intravenous administration of low-viscosity formulations comprising a nucleic acid at a concentration of at least 50 mg/mL.

In some aspects, the present disclosure provides methods of treating a disease comprising administering a low-viscosity formulation of the present disclosure, wherein the disease is selected from thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke (including acute ischemic stroke), Ischemia Reperfusion Injury in solid organ transplantation (IRI, including kidney IRI), cytokine release syndrome (CRS) or Chimeric Antigen Receptor (CAR)-T Cell Related Encephalopathy Syndrome (CRES) or CAR-T neurotoxicity, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies or Solid Tumors.

In some embodiments, the low-viscosity formulation is administered at a dosing regimen that provides improved patient quality of life by requiring a reduced administration volume and/or allowing less-frequent administration.

Thus in one embodiment, provided is a low-viscosity formulation for therapeutic administration to a patient, comprising a nucleic acid; wherein the nucleic acid is present in a concentration of at least 50 mg/mL. In some embodiments, the nucleic acid is present in a concentration between 80 and 400 mg/mL. In some embodiments, the nucleic acid is present in a concentration that is at least 80, 85, 90, 95, or 100 mg/mL. The nucleic acid can be present in a concentration between 100 and 400 mg/mL, or 100 and 300 mg/mL. In some embodiments, the nucleic acid has between 45 and 65 bases and/or a mean molecular weight between 13 and 20 kDa. In certain embodiments, the nucleic acid is predominantly single stranded. Thus preferably, the nucleic acid is at least 70%, 75%, 80%, 85%, 90%, or 95% single stranded. In some embodiments, up 5%, 10%, 15%, 20%, 25%, or up to 30% of the bases in the nucleic acid are paired. In other embodiments, the nucleic acid is up 5%, 10%, 15%, 20%, 25%, or up to 30% double stranded.

In some embodiments, the nucleic acid is present as an alkali metal salt. In certain embodiments, the alkali metal salt is a sodium salt. In specific embodiments, the nucleic acid is predominantly single stranded polydeoxyribonucleotides. In some preferred embodiments, the nucleic acid is predominantly single stranded polydeoxyribonucleic sodium salts. In specific embodiments, the nucleic acid is defibrotide.

For improved patient convenience it is important for injectables to be administered to patients as low-viscosity, hypertonic, isotonic, and/or thixotropic therapeutic formulations. In the case of defibrotide, as the concentration is increased a very small change in concentration results in a large change in viscosity and this variation is further affected by temperature. The current invention allows the concentration to be increased while still meeting the criteria for well-tolerated injectable biologics.

Thus, in one embodiment, the above formulations have a viscosity that is less than 70 centipoise (cP). In one embodiment, the viscosity is between 5 and 65 cP, or 10 and 60 cP. Preferably the viscosity is measured under room temperature conditions, such as from 15° C. to 35° C. More preferably, the viscosity is measured between 18° C. to 25° C. Even more preferably, the viscosity is measured at between 21° C. to 23° C.

In another embodiment, the above formulations have an osmolality of between 240 and 700 mOsm/kg. In other embodiments, the above formulations have an osmolality of between 300 and 500 mOsm/kg. In specific embodiments, the above formulations have a pH between 6.8 and 8.5 or between 7 and 8.

In certain embodiments, the buffer or excipient is a buffering system from pH 6 to pH 8. In some embodiments, the buffer or excipient has a pKa value of from between 5 to 10. In some embodiments, the buffer or excipient is citrate and has pKa values of 3.13, 4.76 and 6.4. In some embodiments, the buffer or excipient is succinic acid and has pKa values of 4.22 and 5.64. In some embodiments, the buffer or excipient is MES buffer and has a pKa value of 6.15. In some embodiments, the buffer or excipient is acetic acid and has a pKa value of 4.76. In some embodiments, the buffer or excipient is phosphoric acid and has a pKa value of 7.22. In some embodiments, the buffer or excipient is tartaric acid and has pKa values of 3.04 and 4.37. In some embodiments, the buffer or excipient is Tris buffer and has a pKa value of 8.072. In some embodiments, the buffer or excipient is HEPES buffer and has a pKa value of 7.564. In some embodiments, the buffer or excipient is lidocaine and has a pKa value of 7.7. In some embodiments, the buffer or excipient is benzyl alcohol and has a pKa value of 15.4. In some embodiments, the buffer or excipient is 0.1% to 2% benzyl alcohol. In some embodiments, the buffer or excipient is an amino acid at a concentration from 5 mM to 200 mM. In some embodiments, the buffer or excipient is an amino acid selected from those listed in Table 1 below (the pKa values and the isoelectronic point, pI, are given for the 20 α-amino acids; pKa1=α-carboxyl group, pKa2=α-ammonium ion, and pKa3=side chain group):

TABLE 1

| Amino acid | pKa₁ | pKa₂ | pKa₃ | pI |
|---|---|---|---|---|
| Glycine | 2.34 | 9.60 | — | 5.97 |
| Alanine | 2.34 | 9.69 | — | 6.00 |
| Valine | 2.32 | 9.62 | — | 5.96 |
| Leucine | 2.36 | 9.60 | — | 5.98 |
| Isoleucine | 2.36 | 9.60 | — | 6.02 |
| Methionine | 2.28 | 9.21 | — | 5.74 |
| Proline | 1.99 | 10.6 | — | 6.30 |
| Phenylalanine | 1.83 | 9.13 | — | 5.48 |
| Tryptophan | 2.83 | 9.39 | — | 5.89 |
| Asparagine | 2.02 | 8.80 | — | 5.41 |
| Glutamine | 2.17 | 9.13 | — | 5.65 |
| Serine | 2.21 | 9.15 | — | 5.68 |
| Threonine | 2.09 | 9.10 | — | 5.60 |
| Tyrosine | 2.2 | 9.11 | — | 5.66 |
| Cysteine | 1.96 | 8.18 | — | 5.07 |
| Aspartic acid | 1.88 | 9.60 | 3.65 | 2.77 |
| Glutamic acid | 2.19 | 9.67 | 4.25 | 3.22 |
| Lysine | 2.18 | 8.95 | 10.53 | 9.74 |
| Arginine | 2.17 | 9.04 | 12.48 | 10.76 |
| Histidine | 1.82 | 9.17 | 6.00 | 7.59 |

Certain buffers or excipients may be used to control the stability, viscosity and/or osmolality. In one embodiment, the above formulations comprise one or more buffers or excipients. In certain embodiments, the excipient is selected from the group consisting of sodium citrate, succinate, sodium chloride, arginine, lysine, lidocaine, or polysorbate-80 (“PS-80”). In some embodiments, the buffer is selected from the group consisting of glycylglycine, histidine, tris(hydroxymethyl)aminomethane (“TRIS”), sodium citrate, or 4-(2-hydroxyethyl)-1-piperazineethanesulfonic acid (“HEPES”) buffer. In one preferred embodiment, the buffer is a dipeptide, such as for example L-Carnosine or glycylglycine. Glycylglycine alone and in and combinations with other excipients improves the solution properties of the formulation by minimizing viscosity and/or osmolality for a given concentration of nucleic acid. Glycylglycine containing formulations manifest solution attributes best optimized to physiologically relevant conditions known to improve tolerability and minimize discomfort upon injection. Thus, in one embodiment, provided is a low-viscosity formulation for therapeutic administration to a patient, comprising a nucleic acid; wherein the nucleic acid is present in a concentration of at least 50 mg/mL; and glycylglycine. In some preferred embodiments, the nucleic acid is defibrotide. Some nucleic acids manifest non-Newtonian shear thinning and thixotropic behavior in liquid formulations, and this behavior is prominently evident in high concentration and/or low-viscosity liquid formulations. Thus, in certain embodiments, provided is a low-viscosity formulation for therapeutic administration to a patient, comprising at least 50 mg/mL of a solution of defibrotide; and glycylglycine. In some embodiments, glycylglycine is present in an amount between 5 and 100 mM. More preferably, glycylglycine is present in an amount between 5 and 60 mM or 10 and 40 mM.

In another embodiment, provided is a low-viscosity formulation for therapeutic administration to a patient, comprising: between 100 and 300 mg/mL of a nucleic acid which contains greater than 70% single stranded, polydisperse polydeoxyribonucleotides having between 45 and 65 bases and a mean molecular weight between 13 and 20 kDa; and an excipient comprising glycylglycine in an amount between 10 and 60 mM. In yet another embodiment, provided is a low-viscosity formulation for therapeutic administration to a patient, comprising: between 120 and 250 mg/mL of a nucleic acid which contains greater than 70% single stranded, polydisperse polydeoxyribonucleotides having a mean length between 45 and 65 bases and a mean molecular weight between 13 and 20 kDa; an excipient comprising glycylglycine in an amount between 10 and 100 mM; and wherein the formulation has a viscosity between 5 and 70 cP, and/or an osmolality of between 240 and 550 mOsm/kg and is suitable for oral or parenteral administration to a patient. In some preferred embodiments, the nucleic acid is defibrotide.

In one embodiment, provided is a low-viscosity formulation for therapeutic administration to a patient, comprising: between 100 and 300 mg defibrotide/mL, comprising greater than 70% single stranded, polydisperse polydeoxyribonucleotides having a mean length between 45 and 65 bases and a mean molecular weight between 13 and 20 kDa; an excipient comprising glycylglycine in an amount between 10 and 100 mM; wherein the formulation has a viscosity between 5 and 70 cP, an osmolality of between 240 and 500 mOsm/kg and is suitable for oral or parenteral administration to a patient.

In one embodiment, provided is a method of parenterally administering a low-viscosity formulation of the disclosure. In some embodiments, the formulation is suitable for subcutaneous administration. In certain embodiments, the formulations comprise a device for subcutaneous delivery including self-administration. In some embodiments, provided is a method of delivering subcutaneously a dose of defibrotide over 5 minutes to 3 hours at between 5 and 50 mL of aqueous fluid. In some embodiments, the dose of defibrotide is administered subcutaneously over 5 minutes, over 10 minutes, over 15 minutes, over 20 minutes, over 25 minutes, over 30 minutes, over 35 minutes, over 40 minutes, over 45 minutes, over 50 minutes, over 55 minutes, over 60 minutes, over 65 minutes, over 70 minutes, over 75 minutes, over 80 minutes, over 85 minutes, over 90 minutes, over 100 minutes, over 105 minutes, over 110 minutes, over 115 minutes, over 120 minutes, over 125 minutes, over 130 minutes, over 135 minutes, over 140 minutes, over 145 minutes, over 150 minutes, over 155 minutes, over 160 minutes, over 165 minutes, over 170 minutes, over 175 minutes, or over 180 minutes at between about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, about 40 mL, about 41 mL, about 42 mL, about 43 mL, about 44 mL, about 45 mL, about 46 mL, about 47 mL, about 48 mL, about 49 mL, or about 50 mL.

In other aspects, provided is a method of orally administering a low-viscosity formulation of the disclosure. In other aspects, provided is a method of nasally administering a low viscosity formulation of the disclosure.

In other aspects, provided herein are methods of making the formulations disclosed herein. In additional aspects, provided are methods of packaging a formulation of the invention. In certain aspects, provided are methods of packaging a formulation of the invention in a device that is capable of subcutaneous administration.

In one embodiment, the above formulations can be used for self-administration by patients. In certain embodiments, the above formulations can be used for administration outside of a hospital setting.

In some embodiments, the condition or disease is hepatic VOD with renal or pulmonary dysfunction following hematopoietic stem-cell transplantation.

Devices for delivering the above formulations are also disclosed herein. The devices are generally configured to deliver, inject, infuse, or release a therapeutically effective amount of a defibrotide formulation within a tissue of the patient. The tissue may be any suitable tissue, but generally includes subcutaneous tissue, muscle, dermal tissue, nasal, bronchial, and/or the peritoneum. In one embodiment, the device is configured to deliver the pharmaceutical formulation through the peritoneum and into the peritoneal cavity of the patient.

In some embodiments the devices are removable and adhered to the patient. In some embodiments the devices are not adhered to the patient.

In some embodiments, the devices for delivering the pharmaceutical formulations described herein to a patient include: a housing, a reservoir coupled to the housing for containing the pharmaceutical formulation, a dispenser in fluid communication with the reservoir and configured for delivery of the pharmaceutical formulation to nasal tissue, and a pump configured to actuate delivery of the pharmaceutical formulation from the reservoir and into the nasal tissue or cavity of the patient. The pharmaceutical formulation generally includes a high concentration of defibrotide. In one embodiment, the high concentration defibrotide formulation also has a low viscosity. Initiation of delivery of the pharmaceutical formulation may be automatic or manual.

In some embodiments, the devices for delivering the pharmaceutical formulations described herein to a patient include: a housing configured to be adhered or removably secured to the patient, a reservoir coupled to the housing for containing the pharmaceutical formulation, a dispensing needle in fluid communication with the reservoir and configured for placement into or through a tissue of the patient, a pump configured to actuate delivery of the pharmaceutical formulation from the reservoir through the dispensing needle and into the tissue or a cavity of the patient, and an injection controller operable to automate delivery of the pharmaceutical formulation by the pump. The pharmaceutical formulation generally includes a high concentration of defibrotide. In one embodiment, the high concentration defibrotide formulation also has a low viscosity.

In other embodiments, the devices are patches for delivering a pharmaceutical formulation to a patient comprising an adhesive for removably securing the patch to the patient, where the pharmaceutical formulation comprises between about 100 mg/mL to about 400 mg/mL of defibrotide, and glycylglycine at a concentration of between about 5 mM and about 60 mM, and where the formulation has a viscosity between about 5 and about 70 cP when measured at between 15° C. and 25° C., and an osmolality between about 240 mOsm/kg and about 700 mOsm/kg. The patch may be configured for intradermal or transdermal delivery. Some patches may include a plurality of dispensing needles for delivering/administering the pharmaceutical formulation. The dispensing needles may be microneedles. In one embodiment, the pharmaceutical formulation is contained within a reservoir of the patch. In another embodiment, the pharmaceutical formulation is contained within the plurality of dispensing needles. In a further embodiment, the pharmaceutical formulation is provided in a coating on the dispensing needles.

The devices disclosed herein are typically wearable by the patient as either an on-body type device or an off-body type device. For example, the on-body device may include a housing that is removably secured to the patient via a suitable adhesive. The off-body device may include a housing that is removably secured to the patient by an article of clothing such as a belt. The off-body device may also include a housing that is not secured to the patient, such as a pre-filled subcutaneous syringe or auto-injector or a nasal spray apparatus. Off-body devices may be preferred by patients having skin sensitivities, patients or who require longer administration times, or patients having high activity levels. In some embodiments, the delivery devices include a reusable unit and a disposable unit. For example, the reusable unit may include any electronics, sensors, or batteries, and the disposable unit may include parts the come into contact with the formulation and the patient (e.g., pump, dispensing needle, adhesive). In other embodiments, the entire delivery device is a single-use disposable unit. The devices are typically packaged in a manner that facilitates self-administration by the patient. In one embodiment, the device is configured to inject the pharmaceutical formulation into the dermal or subcutaneous layer of the skin. Such a device may be automated or manually actuated. In some embodiments, the device or parts of the device may be coated with an agent to prevent potential occlusions. In specific embodiments, the device may be coated with heparin.

In some embodiments, the device for delivering the pharmaceutical formulations described herein to a patient include intravenous delivery devices, including but not limited to syringe pumps.

In some embodiments, the devices for delivering the pharmaceutical formulations described herein to a patient include an off-body prefilled syringe or autoinjector such as those described in U.S. Pat. Nos. 6,805,686 and 5,085,642 both of which are incorporated herein by reference in their entireties.

In some embodiments, the devices for delivering the pharmaceutical formulations described herein to a patient include an off-body spray apparatus which may be used for intranasal or inhalation administration such as those described in U.S. Pat. No. 7,862,536 which is incorporated herein by reference in their entireties.

In some embodiments, the devices for delivering the pharmaceutical formulations described herein to a patient include an off-body nasal spray apparatuses such as those described in U.S. Patent Publication Nos. 2008/0220107 and 2002/0174865 both of which are incorporated herein by reference in their entireties.

In some embodiments, the devices for delivering the pharmaceutical formulations described herein to a patient include an intraocular device such as those described in U.S. Pat. Nos. 4,300,557, 4,712,500, 5,098,443 and 6,881,197 which are incorporated herein by reference in their entireties.

### 4. BRIEF DESCRIPTION OF THE FIGURES

FIG. 1A is a graph showing the viscosity of various formulations as a function of defibrotide concentration using 3 different formulation buffers: sodium citrate (diamonds), glycylglycine (squares) or a mixture of sodium citrate and glycylglycine (triangles).

FIG. 1B is a graph showing the viscosity as a function of temperature of formulations containing sodium citrate (blue diamonds), GlyGly (red squares), or GlyGly and sodium citrate (green triangles).

FIG. 1C is a graph showing viscosity decrease over time in formulations containing 20 mM GlyGly (blue circles), 20 mM GlyGly and 34 mM sodium citrate (orange squares), 20 mM GlyGly and 100 mM sodium succinate (blue triangles) and 20 mM GlyGly and 20 mM sodium chloride (red diamonds).

FIG. 1D is a graph showing the osmolality of various formulations as a function of defibrotide concentration using either sodium citrate (diamonds) or glycylglycine (squares).

FIG. 2A is a graph showing the viscosity of 200 mg/mL defibrotide formulations in the presence of various buffers or excipients.

FIG. 2B is a graph showing the osmolality of 200 mg/mL defibrotide formulations in the presence of various buffers or excipients.

FIG. 3A is a graph showing the osmolality increase as a function of sodium salts.

FIG. 3B is a graph showing the viscosity over time of 180 mg/mL defibrotide formulations in the presence of glycylglycine buffers and sodium citrate solutions (containing 0, 20, 34, 80, or 100 mM sodium citrate).

FIG. 4 is a graph showing the effects of temperature over time on the viscosity of 200 mg/mL defibrotide formulations containing glycylglycine buffer.

FIG. 5A is a graph showing the effects of temperature over time on the osmolality of 200 mg/mL defibrotide formulations containing citrate buffer.

FIG. 5B is a graph showing the effects of temperature over time on the osmolality of 200 mg/mL defibrotide formulations containing glycylglycine buffer.

FIG. 6 is a graph showing the pharmacokinetics of three different 200 mg/mL defibrotide formulations of the invention administered subcutaneously using an animal model in comparison to subcutaneous and intravenous administration of commercially available Defitelio®.

FIG. 7 is a graph showing simulated pharmacokinetic profiles of defibrotide following 4× daily 2-hour infusions of 6.25 mg/kg and 2× daily subcutaneous administration of 18 mg/kg assuming 70% bioavailability.

### 5. DETAILED DESCRIPTION OF THE INVENTION

Defibrotide (CAS number 83712-60-1) is a substance derived from materials of natural origin. It is the sodium salt of relatively low molecular weight polydeoxyribonucleotides which are obtained by extraction from animal mucosa. Defibrotide has a diverse size range and is known to have a mean molecular weight (MW) between 13 and 20 kDa. Defibrotide can be obtained according to U.S. Pat. Nos. 4,985,552 and 5,223,609 and/or presents the physical/chemical characteristics described in the same U.S. Pat. Nos. 4,985,552 and 5,223,609, each of which is incorporated herein by reference. Synthetic defibrotide, presented as phosphodiester oligonucleotides that mimic the therapeutic action of defibrotide are described in US20110092576 which is incorporated herein by reference in its entirety.

Defibrotide has numerous therapeutic applications, including use as an anti-thrombotic agent (U.S. Pat. No. 3,829,567), treatment of peripheral arteriopathies, treatment of acute renal insufficiency (U.S. Pat. No. 4,694,134), and treatment of acute myocardial ischaemia (U.S. Pat. No. 4,693,995). More recently, defibrotide has been used for the treatment and prevention of sinusoidal obstruction syndrome/veno occlusive disease (EU clinical trial EudraCT: 2004-000592-33, US clinical trial 2005-01 (ClinicalTrials.gov identifier: NCT00358501). Patients are treated with a 6.25 mg/kg dose given as a two hour intravenous infusion every six hours until signs and symptoms of VOD are mitigated. As mentioned above, Defibrotide is currently sold under the name Defitelio® as a single vial for injection (commercially available from Gentium S.r.L., Villa Guardia, Italy; see package insert available at dailymed.nlm.nih.gov/dailymed/search.cfm?labeltype=all&query=defibrotide). Defitelio® is prepared as an intravenous infusion by a dilution in 5% Dextrose Injection, USP or 0.9% Sodium Chloride Injection, USP. Intravenous preparation is used within 4 hours if stored at room temperature or within 24 hours if stored under refrigeration. It can be administered for a total of 8 hours over 4 intravenous infusions.

The development of novel defibrotide formulations and/or dosage forms for administration by intravenous (IV), subcutaneous (SC), intramuscular (IM), intradermal (ID) or oral (PO) routes of administration may offer improved quality of life for the patients undergoing treatment. For example, decreasing the frequency from 4 times daily to once or twice daily as well as decreasing the duration of the infusions may offer quality of life improvements to patients while being treated. SC route of administration of defibrotide may offer significant reduction of the time for clinical administration and enable outpatient dosing of the product for as long as needed. Combination products including large volume SC delivery devices can also offer added convenience and faster administration by health-care professionals (HCP), care-givers or even self-administration by the patients. The development of defibrotide low viscosity formulations for administration via an automated injection device (point of use filled, prefilled) using syringes or glass or plastic ampules with or without silicone oil) offer improved quality of life for the patients undergoing treatment. The oral route of administration may be associated with ease of dose preparation and administration, reduced pain and is often preferred by patients. The examples of formulation, drug delivery and dosage forms development studies listed above, focus on improving quality of life and patients' experience while on treatment with defibrotide.

In some embodiments, the combination products of a novel defibrotide formulation and a device may be used for organ perfusion, the flushing and cold storage of organs such as kidney, liver and pancreas at the time of organ removal from the donor in preparation for storage, transportation and eventual transplantation into recipients.

In some embodiments, the route of administration affects the efficacy and/or longevity of the formulations of the present disclosure. In some embodiments, subcutaneous, intramuscular, intradermal, intraocular, buccal, intranasal, inhaled and/or intraperitoneal administration is associated with an extended systemic half-life compared to the same formulation administered intravenously. In some embodiments, subcutaneous administration of the formulation provides lower peak-to-trough ratios of plasma concentrations compared to the same formulation administered intravenously. In some embodiments, subcutaneous administration provides improved efficacy and/or improves the safety profile of the formulation compared to the same formulation administrated intravenously.

### 5.1 Definitions

The following definitions are given for a better understanding of the present invention:

As used herein, the term “nucleic acid” includes “nucleic acids and their salts” and refers to molecules which are comprised of nucleotides, including polymers or large biomolecules composed of nucleotide units linked together in a chain; this includes polynucleotides and oligonucleotides including those comprised of ribose and/or deoxyribose monomers; they can be uniform in size and/or sequence or they can be polydisperse; they can be of any length, including a mixture of different lengths, but some embodiments are generally between 10-400 bases, 20-200 bases, or 45-60 bases long; in some embodiments the mean MW is between 5 and 50 kilodaltons (“kDa), between 13 and 30 kDa, or between 13 and 20 kDa, or between 16 to 20 kDa; they can be single or double stranded, but some embodiments are mostly single stranded polydeoxyribonucleotide salts within the limits stated elsewhere in this application. This also includes DNA sequences that are obtained from the controlled depolymerization of animal intestinal mucosal genomic DNA and, as one embodiment, includes defibrotide.

As used herein, the term “defibrotide” refers to both natural and synthetic sources of defibrotide, including synthetic phosphodiester oligonucleotides as described in US patent application number 20110092576. The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. Nos. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)12-24, (dGP)₁₀₋₂₀, (dPp)₁₃₋₂₆, (dCP)₁₀₋₂₀, where: P=phosphoric radical; dAp=deoxyadenylic monomer; dGp=deoxyguanylic monomer; dTp=deoxythymidinic monomer; dCp=deoxycytidynic monomer; and/or shows the following chemical/physical characteristics: electrophoresis=homogeneous anodic mobility, and/or extinction coefficient, E₁ cm^{1%} at 260±1 nm nm=220±10, and/or E₂₃₀/E₂₆₀=0.45±0.04, and/or coefficient of molar extinction (referred to phosphorous) ε(P)=7.750±500, and/or rotatory power [α]_{D}^{20°}=53°±6; and/or reversible hyperchromicity, indicated as % in native DNA and/or h=15±5.

As used herein, the term “polydeoxyribonucleotide” refers to a polymer whose constituent monomer is a deoxyribonucleotide.

As used herein, the term “oligodeoxyribonucleotide” refers to any oligonucleotide composed of deoxyribose monomers.

As used herein, the term “mean MW” refers to the mean or average molecular weight of the polymer.

The term, “glycylglycine” or “Gly-Gly” or “GlyGly” or “glygly” as used herein, refers to a simple peptide, made of two glycine molecules (glycine is a simple, nonessential amino acid); the dipeptide is used in the synthesis of more complicated peptides. Glycylglycine, an ampholyte, is also sometimes referred to as Diglycine, Diglycocoll, Glycine dipeptide, N-Glycylglycine. It can be made by methods such as those described in CN patent application 101759767 which is incorporated herein by reference in its entirety.

The term, “excipient,” as used herein, refers to any substance that may be formulated with defibrotide and may be included for the purpose of enhancement of the defibrotide in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, defibrotide may be combined with any excipient(s) known in the art that allows tailoring its performance during manufacturing or administration as well as its in vitro and in vivo performance. Many of these excipients may be utilized to tailor the pharmacokinetic profiles of defibrotide formulations.

The term, “buffer” or “buffering agent,” as used herein, refers to a solution which resists changes in the hydrogen ion concentration on the addition of a small amount of acid or base. This includes, for example, a weak acid or base that is used to maintain the pH of a solution near a chosen pH value after the addition of another acidic or basic compound. The function of such buffer or buffering agent is to prevent a change in pH of a solution when acids or bases are added to said solution.

The term, “pH adjusting agent,” as used herein, refers to an acid or base used to alter the pH of a solution to a chosen pH value. The function of such an agent is to alter the pH of a solution to the desired value subsequent to the addition of acidic or basic compounds.

The term, “formulation,” as used herein, refers to compositions for therapeutic use, including, for example, a stable and pharmaceutically acceptable preparation of a pharmaceutical composition or formlation disclosed herein.

The term, “low-viscosity formulation,” as used herein, refers to a formulation which has a viscosity that is less than about 1000 centipoise (cP). Normally viscosity is measured at ambient/room temperatures of (e.g. 15° C. to 35° C.; between 18° C. to 25° C. or between 21° C. to 23° C.) depending on the geographic region and/or weather conditions of the room in which it is being measured.

The term, “aqueous formulation,” as used herein, refers to a water-based formulation, in particular, a formulation that is an aqueous solution.

The term, “high concentration formulation” or “high concentration liquid formulation” or “HCLF” or “high concentration low-viscosity” as used herein, refers to those formulations where the concentration of the nucleic acid is about 80 mg/mL or higher; or about 85 mg/mL or higher; and includes both aqueous and non-aqueous formulations.

The term, “high concentration defibrotide formulations” or “low-viscosity formulation” as used herein, refers to those formulations where the defibrotide concentration is about 80 mg/mL or higher; or about 85 mg/mL or higher; and includes both aqueous and non-aqueous formulations.

The term, “viscosity reducer,” as used herein, refers to a buffer, excipient or other agent which acts to lower or stabilize viscosity, for example, glycylglycine, glycine, sodium citrate, sodium succinate, histidine, TRIS buffer, HEPES buffer, sodium chloride, arginine, lidocaine, benzyl alcohol, polysorbate-80, and/or agents such as a hyaluronidase, including for example PH20, which can help stabilize the formulation. For example, the presence of a viscosity reducer as used herein delays or prevents viscosity increases during storage of the formulation at ambient/room temperatures and/or 4° C.

The term, “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the invention, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “physiologically relevant” as used herein, refers to a measurement, level or amount that is suitable for use in a pharmaceutical, therapeutic or other dosage form to be administered to an animal subject, particularly a human subject.

As used herein, the term “parenteral” refers to any non-oral means of administration. It includes intravenous (i.v. or IV) infusion, IV bolus injection, subcutaneous (s.c. or SC), intradermal (ID), dermal patch, intraocular, intranasal, inhalation, intraperitoneal (IP), and intramuscular (i.m. or IM) injection.

As used herein, the terms “administering” or “administration” are intended to encompass all means for directly and indirectly delivering a compound to its intended site of action.

As used herein, the term “animal” means any animal, including mammals and, in particular, humans.

As used herein, the term “patient” refers to a mammal, particularly a human. Patients to be treated by the methods of the disclosed embodiments include both human subjects and animal subjects (e.g., dog, cat, monkey, chimpanzee, and/or the like) for veterinary purposes. The patients may be male or female and may be any suitable age, e.g., neonatal, infant, juvenile, adolescent, adult, or geriatric.

The terms “treat,” “treating” or “treatment,” and the like as used herein, refers to a method of alleviating or abrogating a disease and/or its attendant symptoms. For example, within the meaning of the present invention, the term “treat” also denotes to arrest, delay the onset (i.e., the period prior to clinical manifestation of a disease) and/or reduce the risk of developing or worsening a disease.

The terms “a” and “an,” when used to modify the ingredient of a composition, such as, active agent, buffering agent, and osmolyte, do not denote a limitation of quantity, but rather denote the presence of at least one of the referenced item. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±7%, ±5%, ±4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 70-70, etc.). Furthermore, all values within a given range may be an endpoint for the range encompassed thereby (e.g., the range 50-80 includes the ranges with endpoints such as 55-80, 50-75, etc.).

### 5.2 Pharmaceutical Formulations Comprising Nucleic Acids

In some embodiments, the present disclosure provides low-viscosity formulations of nucleic acids and their salts. In some embodiments, the present disclosure provides low-viscosity, high concentration liquid formulations (HCLFs) of nucleic acids and their salts for convenient drug delivery to a patient. In particular, nucleic acid compositions which may be administered subcutaneously, intravenously, and/or which may require less frequent dosing than nucleic acid products currently on the market are investigated. In certain embodiments, low-viscosity nucleic acid formulations are self-administered on an out-patient basis. Some formulations of the disclosure have thixotropic and sheer thinning behaviors which are particularly preferred for subcutaneous, intradermal, intraperitoneal, and/or intramuscular administration. Formulations as provided herein offer improved tolerability, patient convenience during treatment and opportunity for outpatient dosing in comparison to currently available commercial nucleic acid formulations.

In some embodiments, the viscosity of high concentration nucleic acid formulations provided herein decreases over time. In certain embodiments, the viscosity and/or fluidity of high concentration nucleic acid formulations provided herein decreases under an increase in shear strain. It should be understood that such properties are preferable for injectables and delivery devices, such as a syringe or preloaded subcutaneous device, in which the strain or shear stress the formulation is exposed to increases as the formulation passes from the barrel of the syringe/device through to the reduced orifice of the needle. In certain embodiments, the nucleic acid is defibrotide.

Formulations of the invention, particularly those comprising defibrotide, may be used for the treatment of numerous conditions including, for example, treatment of peripheral arteriopathies, treatment of acute renal insufficiency, treatment of acute myocardial ischemia, treatment and prevention of Graft versus Host Disease (GvHD), treatment and prevention of Transplant-Associated Thrombotic Microangiopathy (TA-TMA), treatment of Ischemia Reperfusion Injury, such as for example, in solid organ transplantation (Kidney IRI for example), treatment and prevention of cytokine release syndrome (CRS) or Chimeric Antigen Receptor (CAR)-T Cell Related Encephalopathy Syndrome (CRES), and treatment and prevention of sinusoidal obstruction syndrome or VOD. In some embodiments, formulations of the invention, particularly those comprising defibrotide, may be administered to patients who have undergone, are undergoing, or are about to undergo, chemotherapy, stem cell ablation, and/or hematopoietic stem cell transplantation (HSCT). Other uses of defibrotide, methods for its production and testing are described in the following patents, patent applications and articles, each of which is hereby incorporated by reference in its entirety: U.S. Pat. Nos. 3,770,720; 3,829,567; 3,899,481; 4,693,134; 4,693,995; 4,938,873; 4,985,552; 5,081,109; 5,116,617; 5,223,609; 5,646,127; 5,646,268; 5,977,083; 6,046,172; 6,699,985; 6,767,554; 7,338,777; 8,551,967; 8,771,663, US Patent Publication Nos. 20080194506; 20090131362; 20110092576; 20130231470; 20140005256, U.S. patent application Ser. Nos. 14/019,674; 14/323,918; 14/408,272, 16/105,319; 62/656,486; 62/657,161; 62/664,657; and International applications WO 2013/190582, WO 2019/028340, PCT/US2019/064901 and PCT/EP2015/077355. See also Palmer and Boa, Defibrotide. A Review of its pharmacodynamic and pharmacokinetic properties, and therapeutic use in vascular disorders, Drugs, 1993, February; 45(2):259-94; which is incorporated by reference herein. Other references cited throughout are also incorporated by reference in their entireties.

In certain embodiments, the defibrotide to be evaluated by the methods described herein are manufactured by a process such as that described in U.S. Pat. Nos. 4,985,552 and 5,223,609, both of which are hereby incorporated by reference in their entireties. In one preferred embodiment of the invention, defibrotide is a polydeoxyribonucleotide corresponding to the following formula of random sequence:

P1-5,(dAp)12-24,(dGp)10-20,(dTp)13-26,(dCp)10-20

[symbol]

wherein: P=phosphoric radical
dAp=deoxyadenylic monomer
dGp=deoxyguanylic monomer
dTp=deoxythymidylic monomer
dCp=deoxycytidylic monomer

The defibrotide as used herein may have one or more or all of the following chemico-physical properties: electrophoresis=homogeneous anodic mobility; extinction coefficient, E1cm1% at 260±1 nm=220±10; extinction ratio, E230/E260=0.45±0.04; coefficient of molar extinction (referred to phosphorus), ε(P)=7.750±500; rotary power [α]D20°=53°±6; reversible hyperchromicity, indicated as % in native DNA, h=15±5; and a purine:pyrimidine ratio of 0.95±0.5.

In some aspects, the present disclosure provides a nucleic acid formulation with various buffers or excipients, such as those found in Remington, The Science and Practice of Pharmacy (Remington the Science and Practice of Pharmacy) Twenty-Second Edition, 2013 Pharmaceutical Press which is hereby incorporated by reference in its entirety. See especially the monograph on Excipients starting at page 1837. Preferably, the nucleic acid is defibrotide. In some embodiments, a nucleic acid other than defibrotide is used. In some embodiments, the present disclosure provides a low viscosity formulation comprising a nucleic acid. In some embodiments, the present disclosure provides a low viscosity formulation comprising defibrotide.

In some embodiments, the formulation includes a dipeptide buffer (e.g. L-Camosine or glycylglycine). In some embodiments of the disclosure the dipeptide buffer includes glycylglycine, which is a dipeptide of glycine. It is commercially available from supply houses, such as Sigma-Aldrich, and is useful as an excipient for biological systems. In some embodiments of the present invention, glycylglycine is present at concentrations between about 1 mM to about 50 mM. In some embodiments, glycylglycine is present at concentrations between about 5 mM to about 100 mM, about 10 to about 60 mM, or about 10 to about 40 mM. In some embodiments, the glycylglycine is present at a concentration of about 1 mM, about 5 mM, about 10 mM, about 15 mM, about 20 mM, about 25 mM, about 30 mM, about 35 mM, about 40 mM, about 45 mM, or about 50 mM.

In other embodiments, the low-viscosity nucleotide formulation comprises glycine at concentrations between about 5 mM to about 100 mM, about 10 to about 60 mM, or about 10 to about 40 mM.

In further embodiments, the low-viscosity nucleotide formulation comprises benzyl alcohol at concentrations between about 5 mM to about 100 mM, about 10 to about 60 mM, or about 10 to about 40 mM. In some embodiments, the low-viscosity nucleotide formulation comprises benzyl alcohol at concentrations between 0.2-2.0%.

In some embodiments, the low-viscosity nucleotide formulation comprises a viscosity reducer at concentrations between about 5 mM and about 100 mM. In some embodiments, the formulation comprises a viscosity reducer at a concentration of about 5 mM, about 6 mM, about 7 mM, about 8 mM, about 9 mM, 10 mM, about 11 mM, about 12, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, or about 100 mM. In some embodiments, the viscosity reducer is glycylglycine, glycine, sodium citrate or benzyl alcohol.

Other buffers or excipients can be present in the present formulation. In some embodiments, the one or more excipients is a viscosity-reducer. In some embodiments, the low-viscosity pharmaceutical formulation comprises a buffer or excipient selected from sodium citrate, sodium succinate, histidine, TRIS buffer, HEPES buffer, sodium chloride, arginine, lidocaine, amino acids, salts, cyclodextrin and derivatives thereof, Captsiol®, Polyvinylpyrrolidone (PVP), Kolloidon 12 PF, Kolloidon 17PF (BASF), Kolliphor HS 15 (BASF), Macrogol (15) hydroxystearate, polyethylene glycol (15)-hydroxystearate, polyoxyethylated 12-hydroxystearic acid, Solutol HS 15, Benzyl alcohol, and/or polysorbate-80. In some embodiments, the low-viscosity formulation comprises a buffer or excipient so that the nucleic acid is in the form of an alkali metal salt. In some embodiments, the buffer or excipient includes a sodium salt. In some embodiments, the buffer or excipient is sodium citrate, sodium succinate, or sodium chloride.

In some embodiments, the buffer or excipient is sodium citrate, sodium succinate, or sodium chloride at a concentration of less than about 150 mM sodium salt. In some embodiments, the formulation comprises about 1-150 mM sodium salt. In some embodiments, the formulation comprises about 1 mM, about 2 mM, about 3 mM, about 4 mM, about 5 mM, mM, about 6 mM, about 7 mM, about 8 mM, about 9 mM, about 10 mM, about 11 mM, about 12 mM, about 13 mM, about 14 mM, about 15 mM, mM, about 16 mM, about 17 mM, about 18 mM, about 19 mM, about 20 mM, mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM sodium salt. In some embodiments, the formulation comprises about 50-55 mM, about 55-60 mM, about 60-65 mM, about 65-60 mM, about 65-70 mM, about 70-75 mM, about 75-80 mM, about 80-85 mM, about 85-90 mM, about 90-95 mM, about 95-100 mM, about 100-105 mM, about 105-110 mM, about 110-115 mM, about 115-120 mM, about 120-125 mM, about 125-130 mM, about 130-135 mM, about 135-140 mM, about 140-145 mM, or about 145-150 mM sodium salt.

In some embodiments, the formulation comprises sodium citrate. In some embodiments, the formulation comprises less than 60 mM sodium citrate. In some embodiments, the sodium citrate is present at concentrations between about 1 to about 50 mM between about 5 to about 60 mM, about 10 to about 60 mM, or about 10 to about 40 mM. In some embodiments, the concentration of sodium citrate is about a 1 mM, about 5 mM, about 10 mM, about 15 mM, about 20 mM, about 25 mM, about 30 mM, about 35 mM, about 40 mM, about 45 mM, about 50 mM, about 55 mM, or about 60 mM. In some embodiments, the sodium citrate is present at concentrations of about 1 mM, 2 mM, 3 mM, 4 mM, 5 mM, 6 mM, 7 mM, 8 mM, 9 mM, 10 mM, about 11 mM, about 12 mM, about 13 mM, about 14 mM, about 15 mM, mM, about 16 mM, about 17 mM, about 18 mM, about 19 mM, about 20 mM, mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM or about 60 mM.

In some embodiments, the low-viscosity formulation comprises salts such as calcium chloride, magnesium chloride, counterions such as Ca²⁺, Cl⁻, Mg²⁺, hoffmeister series (e.g. F—, SO₄²⁻, HPO₄²⁻, acetate, Cl—, NO₃⁻, Mg²⁺, Li+, Na+, K+, NH₄⁺. In some embodiments, the low-viscosity formulation comprises a salt in a concentration of about 10 mM to about 200 mM or from about 10 mM to about 50 mM, or from about 50 mM to about 150 mM. In some embodiments, the low-viscosity formulation comprises a salt in a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 27 mM, about 28 mM, about 129 mM, about 130 mM, about 131 mM, about 132 mM, about 133 mM, about 34 mM, about 35 mM, about 136 mM, about 137 mM, about 138 mM, about 139 mM, about 140 mM, about 41 mM, about 42 mM, about 143 mM, about 144 mM, about 145 mM, about 146 mM, about 147 mM, about 48 mM, about 48 mM, about 149 mM, about 150 mM, about 151 mM, about 152 mM, about 153 mM, about 54 mM, about 55 mM, about 156 mM, about 157 mM, about 158 mM, about 159 mM, about 160 mM, about 61 mM, about 62 mM, about 163 mM, about 164 mM, about 165 mM, about 166 mM, about 167 mM, about 168 mM, about 169 mM, about 170 mM, about 171 mM, about 172 mM, about 173 mM, about 74 mM, about 75 mM, about 76 mM, about 177 mM, about 178 mM, about 179 mM, about 180 mM, about 81 mM, about 82 mM, about 83 mM, about 184 mM, about 185 mM, about 186 mM, about 187 mM, about 88 mM, about 89 mM, about 90 mM, about 191 mM, about 192 mM, about 193 mM, about 194 mM, about 95 mM, about 96 mM, about 97 mM, about 198 mM, about 199 mM, or about 200 mM.

In some embodiments, the low-viscosity formulation comprises an amino acid. In some embodiments, the amino acid is selected from the group including, but not limited to, methionine, phenylalanine, glycine, proline, and/or serine. In some embodiments, the low-viscosity formulation comprises an amino acid at a concentration between about 5 mM to about 100 mM or about 100 mM to about 200 mM. In some embodiments, the low-viscosity formulation comprises and amino acid at a concentration of about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 111 mM, about 112 mM, about 113 mM, about 114 mM, about 115 mM, about 116 mM, about 117 mM, about 118 mM, about 119 mM, about 120 mM, about 121 mM, about 122 mM, about 123 mM, about 124 mM, about 125 mM, about 126 mM, about 27 mM, about 28 mM, about 129 mM, about 130 mM, about 131 mM, about 132 mM, about 133 mM, about 34 mM, about 35 mM, about 136 mM, about 137 mM, about 138 mM, about 139 mM, about 140 mM, about 41 mM, about 42 mM, about 143 mM, about 144 mM, about 145 mM, about 146 mM, about 147 mM, about 48 mM, about 48 mM, about 149 mM, about 150 mM, about 151 mM, about 152 mM, about 153 mM, about 54 mM, about 55 mM, about 156 mM, about 157 mM, about 158 mM, about 159 mM, about 160 mM, about 61 mM, about 62 mM, about 163 mM, about 164 mM, about 165 mM, about 166 mM, about 167 mM, about 168 mM, about 169 mM, about 170 mM, about 171 mM, about 172 mM, about 173 mM, about 74 mM, about 75 mM, about 76 mM, about 177 mM, about 178 mM, about 179 mM, about 180 mM, about 81 mM, about 82 mM, about 83 mM, about 184 mM, about 185 mM, about 186 mM, about 187 mM, about 88 mM, about 89 mM, about 90 mM, about 191 mM, about 192 mM, about 193 mM, about 194 mM, about 95 mM, about 96 mM, about 97 mM, about 198 mM, about 199 mM, or about 200 mM

In some embodiments, the low-viscosity formulation comprises a cyclodextrin or derivative thereof. In some embodiments, the cyclodextrin is selected from a list including, but not limited to, a (alpha)-cyclodextrin, R (beta)-cyclodextrin, y (gamma)-cyclodextrin, and hydroxypropylbetacyclodextrin. In some embodiments, the low-viscosity formulation comprises a cyclodextrin at a concentration of about 0.5% to about 30%. In some embodiments, the low-viscosity formulation comprises about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.10%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%. In some embodiments, the low-viscosity formulation comprises about 10-15%, about 15-20%, about 20-25% or about 25-30%.

In some embodiments, the low-viscosity formulation comprises a multitude of polymeric structures such as Captsiol® (Ligand Pharmaceuticals). In some embodiments, the low-viscosity formulation comprises Captsiol® at a concentration of about 0.5 to about 50%. In some embodiments, the low-viscosity formulation comprises Captsiol® at a concentration of about 0.5% to about 10%. In some embodiments, the low-viscosity formulation comprises about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the low-viscosity formulation comprises Polyvinylpyrrolidone (PVP), Kolloidon 12 PF, Kolloidon 17PF (BASF), and/or Kolliphor HS 15 (BASF) at a concentration of about 0.5% to about 10%. In some embodiments, the low-viscosity formulation comprises Polyvinylpyrrolidone (PVP), Kolloidon 12 PF, and/or Kolloidon 17PF (BASF) at a concentration of about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In some embodiments, the low-viscosity formulation comprises Kolliphor HS 15 (BASF), Macrogol (15) hydroxystearate, polyethylene glycol (15)-hydroxystearate, polyoxyethylated 12-hydroxystearic acid, Solutol HS 15 at a concentration of about 0.1% to about 10%. In some embodiments, the low-viscosity formulation comprises Kolliphor HS 15 (BASF), Macrogol (15) hydroxystearate, polyethylene glycol (15)-hydroxystearate, polyoxyethylated 12-hydroxystearic acid, Solutol HS 15 at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, or about 10.0%.

In some embodiments, the low-viscosity formulation comprises Benzyl alcohol at a concentration of about 0.1% to about 2.0%. In some embodiments, the low-viscosity formulation comprises Benzyl alcohol at a concentration of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In some embodiments the low-viscosity formulation comprises defibrotide in an amount between about 100 mg/mL to about 400 mg/mL, or about 150 mg/mL to about 250 mg/mL, or about 120 mg/mL to about 200 mg/mL, or about 180 mg/mL to about 200 mg/mL. In some embodiments, the low viscosity formulation comprises defibrotide at about 100 mg/mL, about 101 mg/mL, about 102 mg/mL, about 103 mg/mL, about 104 mg/mL, about 105 mg/mL, about 106 mg/mL, about 107 mg/mL, about 108 mg/mL, about 109 mg/mL, about 110 mg/mL, about 111 mg/mL, about 112 mg/mL, about 113 mg/mL, about 114 mg/mL, about 115 mg/mL, about 116 mg/mL, about 117 mg/mL, about 118 mg/mL, about 119 mg/mL, about 120 mg/mL, about 121 mg/mL, about 122 mg/mL, about 123 mg/mL, about 124 mg/mL, about 125 mg/mL, about 126 mg/mL, about 127 mg/mL, about 128 mg/mL, about 129 mg/mL, about 130 mg/mL, about 131 mg/mL, about 132 mg/mL, about 133 mg/mL, about 134 mg/mL, about 135 mg/mL, about 136 mg/mL, about 137 mg/mL, about 138 mg/mL, about 139 mg/mL, about 140 mg/mL, about 141 mg/mL, about 142 mg/mL, about 143 mg/mL, about 144 mg/mL, about 145 mg/mL, about 146 mg/mL, about 147 mg/mL, about 148 mg/mL, about 149 mg/mL, about 150 mg/mL, about 151 mg/mL, about 152 mg/mL, about 153 mg/mL, about 154 mg/mL, about 155 mg/mL, about 156 mg/mL, about 157 mg/mL, about 158 mg/mL, about 159 mg/mL, about 160 mg/mL, about 161 mg/mL, about 162 mg/mL, about 163 mg/mL, about 164 mg/mL, about 165 mg/mL, about 166 mg/mL, about 167 mg/mL, about 168 mg/mL, about 169 mg/mL, about 170 mg/mL, about 171 mg/mL, about 172 mg/mL, about 173 mg/mL, about 174 mg/mL, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL, about 186 mg/mL, about 187 mg/mL, about 188 mg/mL, about 190 mg/mL, about 191 mg/mL, about 192 mg/mL, about 193 mg/mL, about 194 mg/mL, about 195 mg/mL, about 196 mg/mL, about 197 mg/mL, about 198 mg/mL, about 199 mg/mL, about 200 mg/mL, about 201 mg/mL, about 202 mg/mL, about 203 mg/mL, about 204 mg/mL, about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL, about 216 mg/mL, about 217 mg/mL, about 218 mg/mL, about 219 mg/mL, about 220 mg/mL, mg/mL, about 221 mg/mL, about 222 mg/mL, about 223 mg/mL, about 224 mg/mL, about 225 mg/mL, about 226 mg/mL, about 227 mg/mL, about 228 mg/mL, about 229 mg/mL, about 230 mg/mL, mg/mL, about 231 mg/mL, about 232 mg/mL, about 233 mg/mL, about 234 mg/mL, about 235 mg/mL, about 236 mg/mL, about 237 mg/mL, about 238 mg/mL, about 239 mg/mL, about 240 mg/mL, about 241 mg/mL, about 242 mg/mL, about 243 mg/mL, about 244 mg/mL, about 245 mg/mL, about 246 mg/mL, about 247 mg/mL, about 248 mg/mL, about 249 mg/mL, about 250 mg/mL, about 251 mg/mL, about 252 mg/mL, about 253 mg/mL, about 254 mg/mL, about 255 mg/mL, about 256 mg/mL, about 257 mg/mL, about 258 mg/mL, about 259 mg/mL, about 260 mg/mL, about 261 mg/mL, about 262 mg/mL, about 263 mg/mL, about 264 mg/mL, about 265 mg/mL, about 266 mg/mL, about 267 mg/mL, about 268 mg/mL, about 269 mg/mL, about 270 mg/mL, about 271 mg/mL, about 272 mg/mL, about 273 mg/mL, about 274 mg/mL, about 275 mg/mL, about 276 mg/mL, about 277 mg/mL, about 278 mg/mL, about 279 mg/mL, about 280 mg/mL, about 281 mg/mL, about 282 mg/mL, about 283 mg/mL, about 284 mg/mL, about 285 mg/mL, about 286 mg/mL, about 287 mg/mL, about 288 mg/mL, about 289 mg/mL, about 290 mg/mL, about 291 mg/mL, about 292 mg/mL, about 293 mg/mL, about 294 mg/mL, about 295 mg/mL, about 296 mg/mL, about 297 mg/mL, about 298 mg/mL, about 299 mg/mL, about 300 mg/mL about 301 mg/mL, about 302 mg/mL, about 303 mg/mL, about 304 mg/mL, about 305 mg/mL, about 306 mg/mL, about 307 mg/mL, about 308 mg/mL, about 309 mg/mL, about 310 mg/mL, about 311 mg/mL, about 312 mg/mL, about 313 mg/mL, about 314 mg/mL, about 315 mg/mL, about 316 mg/mL, about 317 mg/mL, about 318 mg/mL, about 319 mg/mL, about 320 mg/mL, about 321 mg/mL, about 322 mg/mL, about 323 mg/mL, about 324 mg/mL, about 325 mg/mL, about 326 mg/mL, about 327 mg/mL, about 328 mg/mL, about 329 mg/mL, about 330 mg/mL, about 331 mg/mL, about 332 mg/mL, about 333 mg/mL, about 334 mg/mL, about 335 mg/mL, about 336 mg/mL, about 337 mg/mL, about 338 mg/mL, about 339 mg/mL, about 340 mg/mL, mg/mL, 341 mg/mL, about 342 mg/mL, about 343 mg/mL, about 344 mg/mL, about 345 mg/mL, about 346 mg/mL, about 347 mg/mL, about 348 mg/mL, about 349 mg/mL, about 350 mg/mL, about 351 mg/mL, about 352 mg/mL, about 353 mg/mL, about 354 mg/mL, about 355 mg/mL, about 356 mg/mL, about 357 mg/mL, about 358 mg/mL, about 359 mg/mL, about 360 mg/mL, about 361 mg/mL, about 362 mg/mL, about 363 mg/mL, about 364 mg/mL, about 365 mg/mL, about 366 mg/mL, about 367 mg/mL, about 368 mg/mL, about 369 mg/mL, about 370 mg/mL, about 371 mg/mL, about 372 mg/mL, about 373 mg/mL, about 374 mg/mL, about 375 mg/mL, about 376 mg/mL, about 377 mg/mL, about 378 mg/mL, about 379 mg/mL, about 380 mg/mL, about 381 mg/mL, about 382 mg/mL, about 383 mg/mL, about 384 mg/mL, about 385 mg/mL, about 386 mg/mL, about 387 mg/mL, about 389 mg/mL, about 390 mg/mL, about 391 mg/mL, about 392 mg/mL, about 393 mg/mL, about 394 mg/mL, about 395 mg/mL, about 396 mg/mL, about 397 mg/mL, about 398 mg/mL, about 399 mg/mL, or about 400 mg/mL.

In some embodiments, the formulation comprises defibrotide in an amount between about 100 mg/mL to about 400 mg/mL, a viscosity reducer at a concentration of between about 5 mM and about 100 mM. In some embodiments, the viscosity reducer is glycylglycine, glycine, sodium citrate or benzyl alcohol. In some embodiments, the low viscosity formulation further comprises sodium citrate. In some embodiments, the sodium citrate is present at a concentration of between about 10 mM to about 34 mM.

In some embodiments, the low-viscosity defibrotide formulation comprises about 180 mg/ml of defibrotide, about 10-100 mM viscosity reducer, and about 10-25 mM sodium citrate. In some embodiments, the low-viscosity defibrotide formulation comprises about 120-200 mg/mL of defibrotide, about 10-100 mM viscosity reducer, and about 10-25 mM sodium citrate, wherein the formulation is formulated for subcutaneous or intravenous delivery to a patient. In some embodiments, the viscosity reducer is glycylglycine, glycine, or sodium citrate.

Other excipients can be added to the present formulations, such as preservatives, salts, or pH adjusting agents.

In some embodiments of the invention, the viscosity of the low-viscosity formulation is between about 1 to about 70 cP. In some embodiments, the viscosity of the low-viscosity formulation is between about 5 cP to about 65 cP, or about 10 cP to about 65 cP. In some embodiments, the viscosity of the low-viscosity formulation is about 5 cP, about 10 cP, about 15 cP, about 20 cP, about 25 cP, about 30 cP, about 35 cP, about 40 cP, about 45 cP, about 50 cP, about 55 cP, about 60 cP, about 65 cP, or about 70 cP.

In some embodiments, the viscosity of the low-viscosity formulation is about 1 cp, about 2 cp, about 3 cp, about 4 cp, about 5 cp, about 6 cp, about 7 cp, about 8 cp, about 9 cp, about 10 cp, about 11 cp, about 12 cp, about 13 cp, about 14 cp, about 15 cp, about 16 cp, about 17 cp, about 18 cp, about 19 cp, about 20 cp, about 21 cp, about 22 cp, about 23 cp, about 24 cp, about 25 cp, about 26 cp, about 27 cp, about 28 cp, about 29 cp, about 30 cp, about 31 cp, about 32 cp, about 33 cp, about 34 cp, about 35 cp, about 36 cp, about 37 cp, about 38 cp, about 39 cp, about 40 cp, about 41 cp, about 42 cp, about 43 cp, about 44 cp, about 45 cp, about 46 cp, about 47 cp, about 48 cp, about 49 cp, about 50 cp, about 51 cp, about 52 cp, about 53 cp, about 54 cp, about 55 cp, about 56 cp, about 57 cp, about 58 cp, about 59 cp, about 60 cp, about 61 cp, about 62 cp, about 63 cp, about 64 cp, about 65 cp, about 66 cp, about 67 cp, about 68 cp, about 69 cp, or about 70 cp.

In some embodiments, the low-viscosity formulation has a pKa value between about 5 to about 10. In some embodiments, the buffer or excipient has a pKa value of about 5, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0.

In certain embodiments, the low-viscosity formulation has a pH of about 6 to about pH 8. In some embodiments, the buffer or excipient has a pH of about 6.0, about 6.1, about, 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments of the invention, viscosity of the low-viscosity formulation decreases overtime. In some embodiments, the viscosity decreases during storage of the formulation. In some embodiments, the viscosity of the low-viscosity formulation decreases over about 1 week to about 5 years. In some embodiments, the viscosity of the low-viscosity formulation decreases over about 1 week, about 2 weeks, about 3 weeks, about 4 weeks, about 5 weeks, about 6 weeks, about 7 weeks, about 8 weeks, about 9 weeks, about 10 weeks, about 12 weeks, about 13 weeks, about 15 weeks, about 16 weeks, about 17 weeks, about 18 weeks, about 19 weeks, about 20 weeks, about 5 months, about 6 months, about 7 months, about 8 months, about 9 months, about 10 months, about 11 months, about 1 year, about 2 years, about 3 years, about 4 years, or about 5 years.

In some embodiments, viscosity of the low-viscosity formulation decreases with an increase in temperature. In some embodiments, viscosity of the low-viscosity formulation decreases with increases in temperature of about 1° C. to about 50°. In some embodiments, viscosity of the low-viscosity formulation decreases with increases in temperature of about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, viscosity of the low-viscosity formulation decreases when stored at a temperature of about −20° C. to about 37° C. In some embodiments, viscosity of the low-viscosity formulation decreases when stored at a temperature of about −20° C., about −19° C., about −18° C., about −17° C., about −16° C., about −15° C., about −14° C., about −13° C., about −12° C., about −11° C., about −10° C., about −9° C., about −8° C., about −7° C., about −6° C., about −5° C., about −4° C., about −3° C., about −2° C., about −1° C., about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C. In some embodiments, viscosity of the low viscosity formulation decreases when stored at room temperature (e.g. from 15° C. to 35° C.).

In some embodiments, the viscosity of the low-viscosity nucleic acid formulation provided herein decreases with decreasing mean molecular weight of the nucleic acid. In some embodiments, the viscosity of the low-viscosity nucleic acid formulation provided herein decreases with decreasing mean molecular weight of the nucleic acid at a given concentration of said nucleic acid. In some embodiments, the viscosity of the low-viscosity nucleic acid formulation provided herein decreases with decreasing mean molecular weight of the nucleic acid at a given concentration of said nucleic acid when viscosity is measured under room temperature conditions, such as from 15° C. to 35° C. In some embodiments, the viscosity decreases under increasing shear, agitation, and/or pressure. In some embodiments, the viscosity of the low-viscosity defibrotide formulation decreases between about 50% to about 85% of its initial (time=TO) viscosity. In some embodiments, the viscosity of the low-viscosity defibrotide formulation decreases by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=TO) viscosity.

In some embodiments, the viscosity decreases during administration of the low-viscosity formulation (e.g. when passing through a needle). In some embodiments, the viscosity of the low-viscosity defibrotide formulation decreases between about 50% to about 85% of its initial (time=TO) viscosity. In some embodiments, the viscosity of the low-viscosity defibrotide formulation decreases by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=TO) viscosity. In some embodiments, the determination of the viscosity of the low-viscosity formulation varies depending on the temperature at which it is measured.

In some embodiments, the viscosity of high concentration nucleic acid formulations provided herein increases with an increase in the mean molecular weight of the nucleic acid. In some embodiments, the viscosity of the low-viscosity defibrotide formulation increases between about 10% to about 100% of its initial (time=TO) viscosity. In some embodiments, the viscosity of the low-viscosity defibrotide formulation increases by about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100% of its initial (time=T0) viscosity.

In preferred embodiments, the viscosity is measured under room temperature conditions, such as from 15° C. to 35° C. More preferably, the viscosity is measured between 18° C. to 25° C. Even more preferably, the viscosity is measured at between 21° C. to 23° C.

In some embodiments, the low-viscosity formulations of the present disclosure have an osmolality between about 200 mOsm/kg and about 1000 mOsm/kg. In some embodiments, the low-viscosity formulations of the present disclosure have an osmolality between about 240 mOsm/kg to about 600 mOsm/kg or about 300 mOsm/kg to about 550 mOsm/kg. In some embodiments, the low-viscosity formulations of the present disclosure have an osmolality of about 200 mOsm/kg, about 210 mOsm/kg, about 220 mOsm/kg, about 230 mOsm/kg, about 240 mOsm/kg, about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, about 360 mOsm/kg, about 370 mOsm/kg, about 380 mOsm/kg, about 390 mOsm/kg, about 400 mOsm/kg, about 410 mOsm/kg, about 420 mOsm/kg, about 430 mOsm/kg, about 440 mOsm/kg, about 450 mOsm/kg, about 460 mOsm/kg, about 470 mOsm/kg, about 480 mOsm/kg, about 490 mOsm/kg, about 500 mOsm/kg, about 510 mOsm/kg, about 520 mOsm/kg, about 530 mOsm/kg, about 540 mOsm/kg, about 550 mOsm/kg, about 560 mOsm/kg, about 570 mOsm/kg, about 580 mOsm/kg, about 590 mOsm/kg, about 600 mOsm/kg, about 610 mOsm/kg, about 620 mOsm/kg, about 630 mOsm/kg, about 640 mOsm/kg, about 650 mOsm/kg, about 660 mOsm/kg, about 670 mOsm/kg, about 680 mOsm/kg, about 690 mOsm/kg, about 700 mOsm/kg, about 710 mOsm/kg, about 720 mOsm/kg, about 730 mOsm/kg, about 740 mOsm/kg, about 750 mOsm/kg, about 760 mOsm/kg, about 770 mOsm/kg, about 780 mOsm/kg, about 790 mOsm/kg, about 800 mOsm/kg, about 810 mOsm/kg, about 820 mOsm/kg, about 830 mOsm/kg, about 840 mOsm/kg, about 850 mOsm/kg, about 860 mOsm/kg, about 870 mOsm/kg, about 880 mOsm/kg, about 890 mOsm/kg, about 900 mOsm/kg, about 910 mOsm/kg, about 920 mOsm/kg, about 930 mOsm/kg, about 940 mOsm/kg, about 950 mOsm/kg, about 960 mOsm/kg, about 970 mOsm/kg, about 980 mOsm/kg, about 990 mOsm/kg, or about 1000 mOsm/kg.

In certain embodiments, the viscosity of the defibrotide formulation decreases during storage up to about 85% of its initial (time=TO) viscosity under room temperature storage conditions. In some embodiments, the viscosity of the low-viscosity defibrotide formulation decreases during storage between about 50% to about 85% of its initial (time=TO) viscosity under room temperature storage conditions. In some embodiments, the viscosity of the low-viscosity defibrotide formulation decreases during storage by about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% or about 85% of its initial (time=TO) viscosity under room temperature storage conditions.

In some embodiments, the present disclosure provides for methods for delivering the formulations of the disclosure. In some embodiments, the formulations of the present disclosure are administered intravenously via a device. In some embodiments, the formulations of the present disclosure are administered intravenously via a device in an out-patient setting (e.g. the patient's home). In certain embodiments, the formulations of the present disclosure are subcutaneously delivered. In some embodiments, formulations of the disclosure are administered subcutaneously by means of a device that can be used by the patient. In some embodiments, the low-viscosity formulation is a defibrotide formulation. In some embodiments, the low-viscosity formulation is Defitelio. In some embodiments, the low-viscosity formulation is a High Concentration Liquid Formulation (HCLF).

Devices for subcutaneous administration may be prefilled, with for example a predefined adult or pediatric dose, or may be used to administer a weight-based dose specific for individual patients. In some embodiments, the patient determines the dose and administers it. In certain embodiments, formulations of the invention are administered subcutaneously by means of a device that is commercially available such as, for example, the FREEDOM60® pump or similar (RMS™ Medical Products). In some specific embodiments, formulations of the invention are administered subcutaneously in less than about two hours, less than about one hour, or less than about 30 minutes. In some specific embodiments, formulations of the invention are delivered subcutaneously over about 5 minutes to about 1 hour, about 10 minutes to about 1 hour or about 15 minutes to about 45 minutes. In some embodiments, the low-viscosity formulations are delivered subcutaneously over about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 165 minutes, about 170 minutes, about 175 minutes, about 180 minutes, about 185 minutes, about 190 minutes, about 200 minutes, about 205 minutes, about 210 minutes, about 215 minutes, about 220 minutes, about 225 minutes, about 230 minutes, about 235 minutes, about 240 minutes, about 245 minutes, about 250 minutes, about 260 minutes, about 265 minutes, about 270 minutes, about 275 minutes, about 280 minutes, about 285 minutes, about 290 minutes, about 295 minutes, about 300 minutes, about 305 minutes, about 310 minutes, about 315 minutes, about 320 minutes, about 325 minutes, about 330 minutes, about 335 minutes, about 340 minutes, about 345 minutes, about 350 minutes, about 355 minutes, or about 360 minutes. In some embodiments, the infusion delivers a consistent concentration of the low viscosity formulation. In some embodiments, the infusion delivers a varied concentration of the low-viscosity formulation. In some embodiments, the initial concentration of the low-viscosity formulation is higher than the concentration delivered later in the regimen. In some embodiments, the initial concentration of the low-viscosity formulation is lower than the concentration delivered later in the regimen. In some embodiments, a bolus of the low-viscosity formulation is delivered before infusion of the same formulation begins. In some embodiments, the concentration of the low-viscosity formulation administered via bolus is greater than the concentration of the low-viscosity formulation administered via infusion.

In some embodiments, the low-viscosity formulation given subcutaneously exhibits improved Cmax and/or AUC compared to an intravenously administered defibrotide formulation. In some embodiments, the low-viscosity formulation administered subcutaneously exhibits a Cmax about 5% to about 500% greater than the Cmax exhibited by intravenously administering the low-viscosity formulation. In some embodiments, the low-viscosity formulation administered subcutaneously exhibits Cmax about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 410%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% or more greater than the Cmax exhibited by intravenously administering the low-viscosity formulation.

In some embodiments, the low-viscosity formulation administered subcutaneously exhibits a AUC about 5% to about 500% greater than the AUC exhibited by intravenously administering the low-viscosity formulation. In some embodiments, the low-viscosity formulation administered subcutaneously exhibits AUC about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84% about 85%, about 86%, about 87%, about 88%, about 89%, about 90%, about 91%, about 92%, about 93%, about 94%, about 95%, about 96%, about 97%, about 98%, about 99%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% or more greater than the AUC exhibited by intravenously administering the low-viscosity formulation.

The formulation dosing may be determined by a variety of factors that will be readily apparent to a skilled artisan. In some embodiments, the dose is based on patient's baseline body weight. In some embodiments, formulation is administered in an amount of about 1 to about 100 mg per kilogram of body weight per day. For example the formulation is administered in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg per kilogram of body weight per day. In some embodiments, formulation is administered in an amount of about 25 mg per kilogram of body weight per day. In some embodiments, doses based on the patient's body weight are rounded to the nearest 10 mg for patients over 35 kg. In some embodiments, doses based on the patient's body weight were rounded to the nearest 5 mg for patients under 35 kg. In some embodiments, the formulation is a defibrotide formulation.

The formulation may be administered as a single daily dose or in multiple doses per day. In some embodiments, formulation is administered once a day. In some embodiments, formulation is administered in multiple doses per day. For example, the formulation may be administered in 2, 3, 4, 5, 6, 7, 8, 9, or in 10 doses per day. In some embodiments, the formulation is administered in four doses per day. In some embodiments, the formulation is administered in four doses per day every 6 hours.

In some embodiments, the dose and frequency of administration varies depending on route of administration. In some embodiments, subcutaneous administration of the low-viscosity formulations of the present disclosure allows for less-frequent administration and/or lower doses. In some embodiments, subcutaneous administration of the low-viscosity formulation of the present disclosure allows for reduced administration volume.

In some embodiments, the dose of defibrotide is subcutaneously administered over 5 minutes to 3 hours at between 5 and 50 mL of aqueous fluid. In some embodiments, the dose of defibrotide is administered subcutaneously over 5 minutes, over 10 minutes, over 15 minutes, over 20 minutes, over 25 minutes, over 30 minutes, over 35 minutes, over 40 minutes, over 45 minutes, over 50 minutes, over 55 minutes, over 60 minutes, over 65 minutes, over 70 minutes, over 75 minutes, over 80 minutes, over 85 minutes, over 90 minutes, over 100 minutes, over 105 minutes, over 110 minutes, over 115 minutes, over 120 minutes, over 125 minutes, over 130 minutes, over 135 minutes, over 140 minutes, over 145 minutes, over 150 minutes, over 155 minutes, over 160 minutes, over 165 minutes, over 170 minutes, over 175 minutes, or over 180 minutes at between about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 21 mL, about 22 mL, about 23 mL, about 24 mL, about 25 mL, about 26 mL, about 27 mL, about 28 mL, about 29 mL, about 30 mL, about 31 mL, about 32 mL, about 33 mL, about 34 mL, about 35 mL, about 36 mL, about 37 mL, about 38 mL, about 39 mL, about 40 mL, about 41 mL, about 42 mL, about 43 mL, about 44 mL, about 45 mL, about 46 mL, about 47 mL, about 48 mL, about 49 mL, or about 50 mL.

In some embodiments, the dose of defibrotide is subcutaneously administered over about 120 minutes at a volume of about 50 mL. In some embodiments, the dose of defibrotide is subcutaneously administered over about 20 minutes at a volume of about 5 mL. In some embodiments, the dose of defibrotide is about 20 mg administered subcutaneously over 20 minutes at a volume of about 5 mL. In some embodiments, the dose of defibrotide is about 20 mg administered subcutaneously over 20 minutes at a volume of about 5 mL administered four times a day.

In some embodiments, the dose of defibrotide is intravenously administered. In some embodiments, the dose of defibrotide is intravenously administered to patients by a 30 minute to a 6 hour infusion. In some embodiments, the dose of defibrotide is intravenously administered to patients by a 30 minute to a 6 hour infusion every 1 to 24 hours. In some embodiments, the dose of defibrotide is intravenously administered to patients by 2-hour intravenous infusions every 6 hours. In some embodiments, the dose of defibrotide is intravenously administered to patients by 2-hour intravenous infusions every 6 hours for a minimum of 10-40 days. In some embodiments, the dose of defibrotide is intravenously administered to patients by 2-hour intravenous infusions every 6 hours for a minimum of 21 days.

As a skilled artisan will appreciate, the treatment period may vary on a patient-by-patient basis. In some embodiments, the treatment period is determined by monitoring signs and symptoms of the disease being treated. In some embodiments, the low-viscosity formulations of the present disclosure are administered until patient signs and symptoms of the disease being treated are decreased, ameliorated, delayed, or treated. In some embodiments, the low-viscosity formulations of the present disclosure are administered until patient signs and/or symptoms of the disease being treated are decreased, ameliorated, and/or delayed by about 1% to about 100% compared to the signs and symptoms of the disease in the same patient before treatment or an untreated patient. In some embodiments, the low-viscosity formulations of the present disclosure are administered until patient signs and symptoms of the disease being treated are decreased, ameliorated, delayed by at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% compared to the signs and symptoms of the disease in the same patient before treatment or an untreated patient. For example, in some embodiments, the treatment period is determined by monitoring signs and symptoms of hepatic VOD. For example, if the signs and symptoms of hepatic VOD are still present after an initial treatment period, defibrotide treatment is continued until resolution of VOD. In some embodiments, if the signs and symptoms of hepatic VOD are still present after 21 days, defibrotide treatment is continued until resolution of VOD up to a maximum of 60 days. Thus, in certain embodiments, the treatment period may last anywhere from 21 to 60 days. For example, the treatment period lasts for 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the treatment period lasts 21 days.

In some embodiments, administration of the formulations of the present disclosure treats or ameliorates development of VOD and/or VOD symptoms compared to an untreated patient or the same patient before formulation administration. In some embodiments, VOD and/or VOD symptoms are treated or ameliorated in the patient between day 1 and year 10. In some embodiments, administration of the formulation treats or ameliorates development of VOD and/or VOD symptoms compared to an untreated patient or the same patient before defibrotide administration at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration for about 1 day, about 1 week, about 1 month, about 2 months, about 3 months, about 4 months, about 5 months, about 6 months, about 1 year, about 2 years, about 5 years, or about 10 years, or more.

In some embodiments, administration of the formulations of the present invention prevents VOD and/or VOD symptoms.

In some embodiments, administration of the formulations of the present disclosure treats or ameliorates VOD and/or VOD symptoms by about 1%, about 5%, about 10%, about 20%, about 30%, about 40%, about 50%, about 60%, about 70%, about 80%, about 90%, or about 100% compared to an untreated patient or the same patient before formulation administration. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% at about day 1, about day 2, about day 3, about day 4, about day 5, about day 6, about week 1, about week 2, about week 3, about week 4, about week 5, about week 6, about week 7, about week 8, about week 9, about week 10, about week 20, about week 30, about week 40, about week 50, about week 60, about week 70, about week 80, about week 90, about week 100, about year 1, about year 2, or about year 3. In some embodiments, administration of the formulation treats or ameliorates development of VOD and/or VOD symptoms compared to an untreated patient or the same patient before formulation administration by about 1%, about 5%, about 10%, about 20%, about 30%, about 40%, about 50%, about 60%, about 70%, about 80%, about 90%, or about 100% for about 1, about 2 days, about 3 days, about 4 days, about 5 days, about 6 days, about 1 week, about 2 weeks, about 3 weeks, about 4 weeks, about 1 month, about 2 months, about 3 months, about 4 months, about 5 months, about 6 months, about 1 year, about 2 years, about 5 years, or about 10 years or more.

### 5.3 Exemplary Delivery Devices

Defibrotide is typically administered to patients in the hospital by 2-hour intravenous infusions every 6 hours for a minimum of 21 days, as previously mentioned. Given the need for frequent IV administration, this inpatient treatment regimen is inconvenient. The devices described herein may provide a more convenient option for administration of the defibrotide formulations also described herein.

The devices are generally configured to deliver inject, infuse, or release a therapeutically effective amount of a defibrotide formulation within a patient's tissue. For example, the devices may deliver the defibrotide formulation into a patient's circulation, subcutaneous tissue, muscle, dermal tissue, and/or the peritoneum. In one embodiment, the device is configured to deliver the pharmaceutical formulation through the peritoneum and into the peritoneal cavity of the patient. Delivery may be accomplished in any suitable manner, e.g., by injection, infusion, drip, etc., so long as the pharmaceutical formulation is delivered to the intended tissue.

The devices are typically wearable by the patient as either an on-body type device or off-body type device. For example, the on-body device may include a housing that is removably secured to the patient via a suitable adhesive. The off-body device may include a housing that is removably secured to the patient by an article of clothing such as a belt or hanging as a lanyard. Off-body devices include a tube set for the connection to the patient. Tube sets are commercially available. Off-body devices may be preferred by patients having skin sensitivities, patients or who require longer administration times, or patients having high activity levels. In some embodiments, the delivery devices include a reusable unit and a disposable unit. For example, the reusable unit may include any electronics, sensors, or batteries and the disposable unit may include parts the come into contact with the formulation and the patient (e.g., pump, dispensing needle, adhesive). In other embodiments, the entire delivery device is a single-use disposable unit. The devices are typically packaged in a manner that facilitates self-administration by the patient.

In some embodiments, commercially available devices or drug delivery platforms as well as those in development may be used to subcutaneously deliver the defibrotide formulations. Exemplary devices and drug delivery platforms include but are not limited to, those being developed by the following companies: Sensile Medical (SenseCore™ pump) (Boonton, N.J.), West Pharmaceuticals (SmartDose@ self-injection platform) (Exton, Pa.), YpsoMed (YpsoDose® injector) (Burgdorf, Switzerland), Cane S.p.A (Crono® infusion pump) (Turin, Italy), Unilife Corporation (Precision-Therapy™ injection device and Flex-Therapy™ injection device) (York, Pa.), and SteadyMed Therapeutics (PatchPump® infusion system) (San Ramon, Calif.).

In other embodiments, the devices may deliver the defibrotide formulation intramuscularly, intradermally, transdermally, nasally, orally, or intraperitoneally to the patient. Nasal delivery devices include devices that aspirate, aerosolize, or otherwise aid in nasal delivery of a pharmaceutical formulation of the disclosure.

Intradermal and transdermal variations of the device may include a patch. In one embodiment, the patch includes an adhesive for removably securing the patch to the patient, where the pharmaceutical formulation comprises between about 100 mg/mL to about 400 mg/mL of defibrotide, and a viscosity reducer at a concentration of between about 5 mM and about 100 mM, and where the formulation has a viscosity between about 5 and about 70 cP when measured at between 15° C. and 25° C., and an osmolality between about 240 mOsm/kg and about 1000 mOsm/kg. Preferably, the viscosity reducer is glycylglycine, glycine, sodium citrate, benzyl alcohol or hyaluronidase (PH20). Some patches may include a plurality of dispensing needles for delivering/administering the pharmaceutical formulation. The dispensing needles may be microneedles. In one embodiment, the pharmaceutical formulation is contained within a reservoir of the patch. In another embodiment, the pharmaceutical formulation is contained within the plurality of dispensing needles. In a further embodiment, the pharmaceutical formulation is provided in a coating on the dispensing needles. Exemplary needle patches are the Micro-Trans™ microneedle array patch (Valeritas, Bridgewater Township, N.J.) and AdminPatch® microneedle array (AdminMed, Sunnyvale, Calif.). In some embodiments the patches may be biodegradable or include biodegradable needles. For example, the adhesive portion of the patch may be non-biodegradable and the needles may be biodegradable. In other embodiments, the pharmaceutical formulation is delivered to the patient solely via one or more biodegradable needles (without a patch). In yet further embodiments the pharmaceutical formulation is delivered to the patient solely via non-biodegradable needles, e.g., with metal needles or projections (without a patch).

In further embodiments, the device is a handheld injection device. Injection may be accomplished by any projection or protrusion capable of disrupting the surface of the skin. The handheld injection device may be an intradermal injection device or one configured to inject the pharmaceutical formulation into subcutaneous tissue. The handheld injection device may be automated or manually actuated. In these embodiments, the injection needle (dispensing needle) of the handheld injection device may be about 1.0 mm to about 5 mm. However, in some embodiments the injection needle may be less than 1.0 mm. The injection needles may be solid or hollow. Exemplary handheld injection devices include without limitation, the MicronJect intradermal injector (Nanopass Technologies, Ltd., Nes Ziona, Israel), the Macroflux® system (Zosano Pharma, Fremont, Calif.), VAX-ID™ intradermal injector (Novosanis, Belgium), and MTS® microneedle drug delivery system (3M, St. Paul, Minn.)). The handheld injection device or handheld injector may also be configured to inject the pharmaceutical formulation into the peritoneum or through the peritoneum and into the peritoneal cavity of the patient. Another device for peritoneal delivery may include one or more infusion catheters attached to a reservoir attached to the patient's body as a wearable device, or one or more infusion catheters attached to a reservoir that is not worn by the patient, but which is portable so that the patient can receive treatment in an ambulatory setting.

In some embodiments, the device lacks a dispensing needle and delivers the pharmaceutical formulation into dermal, subcutaneous, or muscle tissue layers by injecting the formulation under high pressure (jet injectors). Exemplary jet injectors for use in delivering the defibrotide formulation include without limitation, the Biojector® device and the ZetaJet™ needle free injection system (iHealthNet, Alpharetta, Ga.) the Stratis Needle-Free injector (PharmaJet, Golden, Colo.). Other gas or spring-powered injectors may also be used.

Devices for delivery/administration of the defibrotide formulation may be prefilled, with for example a predefined adult or pediatric dose, or may be used to administer a weight-based dose specific for individual patients. In some embodiments, the patient determines the dose and administers it. In certain embodiments, formulations of the invention are administered subcutaneously by means of a device that is commercially available such as, for example, the FREEDOM60® pump or similar (RMS™ Medical Products). Other exemplary administration devices are mentioned above. In some specific embodiments, formulations of the invention are administered subcutaneously in less than about two hours, less than about one hour, or less than about 30 minutes. In some specific embodiments, formulations of the invention are delivered subcutaneously over about 5 minutes to about 1 hour, about 10 minutes to about 1 hour or about 15 minutes to about 45 minutes.

In some embodiments, the devices for delivering the pharmaceutical formulations described herein to a patient include: a housing configured to be removably secured to the patient, a reservoir coupled to the housing for containing the pharmaceutical formulation, a dispensing needle in fluid communication with the reservoir and configured for placement into or through a tissue of the patient, a pump configured to actuate delivery of the pharmaceutical formulation from the reservoir through the dispensing needle and into the tissue or a cavity of the patient, and an injection controller operable to automate delivery of the pharmaceutical formulation by the pump. The pharmaceutical formulation generally includes a high concentration of defibrotide. In one embodiment, the high concentration defibrotide formulation also has a low viscosity. In another embodiment, the pharmaceutical formulation includes between 80 mg/mL to about 400 mg/mL of defibrotide, and a viscosity reducer at a concentration of between about 5 mM and about 350 mM, where the pharmaceutical formulation has a viscosity between about 5 and about 70 cP when measured at between 15° C. and 25° C., and an osmolality between about 240 mOsm/kg and about 700 mOsm/kg.

The delivery devices are generally designed to be worn by the patient. Thus, they may be sized and shaped to suit the particular body area for intended attachment, coupling, or securement. For example, the devices may be shaped as a circle, oval, square, rectangle, triangle, or any combination thereof. The devices may be removably secured to the patient in a variety of ways. In embodiments where the device is directly attached to the skin of the patient (“on-body”), any suitable adhesive may be used. The adhesive may be placed on the surface of the device housing that is to face the patient. In embodiments where the device is not directly attached to the patient's skin (“off-body”), the device or a portion thereof (e.g., the housing) may be removably secured to the patient using a belt, pouch, or other article of clothing.

A reservoir for containing the pharmaceutical formulation is generally coupled to the housing. The reservoir may be integrally formed with the housing (e.g., formed within the housing), or it may be configured for insertion into the housing or attachment to the housing. Reservoirs may comprise syringes, vials, cartridges, and the like. In one embodiment, the reservoir is a cartridge capable of placement into the housing. In another embodiment, the reservoir is a vial capable of placement into the housing. In yet another embodiment, the reservoir is a syringe capable of placement into the housing. In a further embodiment, the reservoir is a syringe that is attached to the housing, e.g., by a luer type connector, friction fit, a screw-type fitting, a clamp, etc. In some embodiments, a plurality of reservoirs are attached to the housing. In other embodiments, the reservoirs are pre-filled with the pharmaceutical formulation and thus contain the pharmaceutical formulation prior to placement within the housing or attachment to the housing. After delivery of the pharmaceutical formulation is completed, the reservoir may be removed or detached from the housing, or replaced with another reservoir.

The devices described herein generally include a dispensing needle in fluid communication with the reservoir, and which is configured for placement into or through a tissue of the patient. The tissue may be subcutaneous tissue, muscle, dermal tissue, or the peritoneum of the patient, as mentioned above. The dispensing needle may be made from any metallic or polymeric material having suitable strength to penetrate the skin to the subcutaneous layer. The length and diameter (both inner and outer diameter) of the dispensing needle may also be adapted for subcutaneous administration. In some embodiments, the dispending needle is configured to reduce side-effects near the area of needle entry such as redness (erythema), swelling, and induration. For example, a side port may be provided near the distal end of the dispensing needle to provide irrigation fluid to the tissue. In some embodiments, the dispensing needle may include a multi-beveled tip, e.g., three or five bevels. In yet further embodiments the dispensing needle may include a tip with perforations configured to generate a spray within the tissue. Some variations of the device may have the dispensing needle connected to the reservoir and/or housing via tubing.

The devices described herein also include a pump that actuates flow or delivery of the pharmaceutical formulation from the reservoir through the dispensing needle and into the tissue or a cavity, e.g., the peritoneal cavity, of the patient. The pump may be any pump suitable for wearable drug delivery devices. For example, the pump may be a positive displacement pump. In one embodiment, the positive displacement pump includes a rotating piston. In another embodiment, the positive displacement pump includes a slidable piston. In yet further embodiments, the pump is an osmotic pump. In this embodiment, osmosis may be used to generate the pumping force. For example, the pump may include two chambers of liquid separated by a semi-permeable membrane. Adding salt to one chamber draws in liquid from the other chamber. The resulting hydraulic/mechanical pressure generated by the liquid movement drives a plunger to corresponding move and push drug out of a reservoir.

The injection controller of the device generally implements operating parameters of the device that control and automate delivery of the pharmaceutical formulation. For example, the injection controller may include a processor for executing the operating parameters. As further described below, the operating parameters can be stored in memory of the injection controller or be wirelessly received from a handheld controller.

In some embodiments, the device may be part of system configured for wireless communication of information between a handheld controller and the housing or injection controller. In these embodiments, the housing or injection controller may include an antenna for wirelessly receiving information from the handheld controller. Any suitable type of antenna may be employed. For example, the antenna may be a ring or coil, and be configured for radiative coupling, inductive coupling, magnetic induction, etc. The wirelessly received information may include information about delivery of the pharmaceutical formulation selected from the group consisting of flow rate, pump speed, and duration of delivery. In one embodiment, information and power (e.g., if the housing does not include a battery) is delivered to the housing via the antenna. In a further embodiment, the handheld controller also includes an antenna for receiving information back from the housing or injection controller. The handheld device may be used by the patient and/or a physician to manually control or adjust formulation delivery from the device. Alternatively, instructions stored in memory can be run by a processor in the handheld device to control formulation delivery. The instructions can provide for the automated delivery of the formulation. In some embodiments, manual input or manual control can override the automated delivery information/instructions.

In some embodiments, the delivery device comprises a patch configured for removable securement to a patient. The patch may be an adhesive patch including one or more layers. The layers may be formed from one or more polymeric materials. When a patch is used, one or more of the patch layers may be formed to include the defibrotide formulation and thus function as a reservoir. In some instances, the reservoir may be disposed between the layers or on a surface of an outermost layer of the patch. The patches may deliver the pharmaceutical formulation in a sustained release, immediate release, or delayed release fashion, or any combination of defibrotide release may be employed. The patches may be of any suitable size and shape, may include any number of layers, and may have a varying amount of flexibility.

In one embodiment, the patch is a transdermal patch configured to deliver the pharmaceutical formulation to the dermal and/or subcutaneous tissue of the patient. The use of an occlusive top layer on the patch or application of heat over the patch after it is placed on the patient's skin may facilitate delivery to the dermal, subcutaneous, or other skin layer. One or more penetration enhancers may also be included in the transdermal patch to aid in delivering the pharmaceutical formulation. Exemplary penetration enhancers include without limitation, fatty alcohols, fatty acids (linear or branched), terpenes (e.g., mono, di and sequiterpenes; hydrocarbons, alcohols, ketones), fatty acid esters, organic acids, ethers, amides, amines, hydrocarbons, alcohols, phenols, polyols, and surfactants (e.g., anionic, cationic, nonionic, and bile salts).

In further embodiments, the patch may comprising a plurality of dispensing needles, e.g., a plurality of microneedles. Such a needle patch may include various layers and include a reservoir between the layers, or the needles could function as the reservoir and contain a small volume of the defibrotide formulation. Accordingly, the dose of the defibrotide formulation can be adjusted based on the number of dispensing needles employed. The needle patches may be of any suitable shape and size, may have any suitable amount of flexibility, and may have any suitable number of dispensing needles. The patch and dispensing needles may be made from the same or different materials. For example, the patch may be made from a polymeric material and the dispensing needles may be made from a metal such as stainless steel. Alternatively, the patch and dispensing needles may both be made from a polymeric material. The length of the dispensing needles may also be adjusted based on the type of delivery. For example, although the length of the dispensing needles may be set for intradermal delivery, the length may be adjusted in some instances for subcutaneous delivery.

The pharmaceutical formulation delivered by the devices described herein generally include defibrotide. In some embodiments, a high concentration of defibrotide is used in the pharmaceutical formulation. The high concentration defibrotide formulations may also have a low viscosity.

In one embodiment, the devices deliver a pharmaceutical formulation including between about 100 mg/mL to about 400 mg/mL of defibrotide, and a viscosity reducer at a concentration of between about 5 mM and about 60 mM, where the formulation has a viscosity between about 5 and about 70 cP when measured at between 15° C. and 25° C., and an osmolality between about 240 mOsm/kg and about 700 mOsm/kg. In another embodiment, the pharmaceutical formulation also includes a buffer or excipient selected from sodium citrate, sodium succinate, histidine, TRIS buffer, HEPES buffer, sodium chloride, arginine, lidocaine, and/or polysorbate-80. In a further embodiment, the pharmaceutical formulation includes a buffer or excipient so that the nucleic acid of the formulation is in the form of an alkali metal salt. In some embodiments, the buffer or excipient is sodium citrate, sodium succinate, or sodium chloride. In other embodiments, the buffer or excipient is sodium citrate, sodium succinate, or sodium chloride at a concentration of less than about 80 mM sodium salt. In one embodiment, the buffer or excipient is sodium citrate at a concentration of between about 20 mM and about 34 mM. The pharmaceutical formulation may be delivered subcutaneously, intramuscularly, intradermally, transdermally, orally, nasally, and/or intraperitoneally.

Various properties of the defibrotide formulation delivered by the devices may change over time. In one embodiment, the viscosity of the defibrotide formulation decreases over time. In another embodiment, the viscosity of the defibrotide formulation decreases during storage. In a further embodiment, the viscosity of the defibrotide formulation decreases under increasing shear, agitation, temperature, time, and/or pressure. In one embodiment, the shear increases during administration. In some embodiments, shear increases during administration via the dispensing needle.

The pharmaceutical formulations delivered subcutaneously by the devices may also demonstrate an extended systemic half-life compared to a defibrotide formulation not comprising a viscosity reducer. In one embodiment, the devices subcutaneously deliver a pharmaceutical formulation that exhibits lower peak-to-trough ratios of plasma concentrations compared to an intravenously administered defibrotide formulation. In some embodiments, the low viscosity defibrotide formulation given subcutaneously exhibits improved Cmax and/or AUC compared to an intravenously administered defibrotide formulation. In another embodiment, the devices subcutaneously deliver a pharmaceutical formulation that exhibits improved efficacy and/or an improved safety profile compared to a defibrotide formulation not comprising a viscosity reducer. The devices may deliver a defibrotide formulation that is hypertonic, isotonic or thixotropic.

Methods for delivering the above pharmaceutical formulations are also described herein. In some embodiments, the methods include delivering the pharmaceutical formulations to patients using any one of the aforementioned devices, where the route of delivery is subcutaneous, intramuscular, intradermal, transdermal, oral, nasal, and/or intraperitoneal (e.g., into or through the peritoneum). For example, the device may be an on-body device, and off-body device, a device wirelessly controlled via a handheld controller, a device with a pre-filled reservoir, etc. The pharmaceutical formulation delivered by the devices may be used to treat or prevent a disease or a condition including, but not limited to, thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury, such as for example, in solid organ transplantation (Kidney IRI for example), treatment and prevention of cytokine release syndrome (CRS) or Chimeric Antigen Receptor (CAR)-T Cell Related Encephalopathy Syndrome (CRES); treatment and prevention of sinusoidal obstruction syndrome or VOD, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies, and Solid Tumors. Furthermore, the dosing regimen implemented by the device will generally be tailored to improve patient quality of life by requiring a reduced administration volume and/or allowing less-frequent administration and/or a shorter duration of administration.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the and scope of the invention.

### EMBODIMENTS

Embodiment 1: A device for delivering a pharmaceutical formulation to a patient comprising:

(a) a housing;
(b) a reservoir coupled with the housing for containing the pharmaceutical formulation;
(c) a dispenser in fluid communication with the reservoir and configured for delivery of the pharmaceutical formulation into or through a tissue or a cavity of the patient;
(d) a pump or spring configured to actuate delivery of the pharmaceutical formulation from the reservoir through the dispensor and into the tissue or a cavity of the patient; and
(e) a controller operable to deliver the pharmaceutical formulation.

Embodiment 2: The device of embodiment 1, wherein the pharmaceutical formulation comprises between about 50 mg/mL to about 400 mg/mL of defibrotide.

Embodiment 3. The device of embodiment 1 or 2, wherein the formulation further comprises a viscosity reducer at a concentration of between about 5 mM and about 350 mM, and wherein the formulation has a viscosity between about 5 and about 70 cP when measured at between 15° C. and 25° C., and an osmolality between about 240 mOsm/kg and about 1000 mOsm/kg.

Embodiment 4. The device of any of embodiments 1-3, wherein the housing is configured to be off-body or removably secured to the patient

Embodiment 5: The device of any of embodiments 1-4, further comprising an adhesive that removably secures the housing to the patient.

Embodiment 6: The device of any of embodiments 1-5, further comprising an article of clothing that removably secures the housing to the patient.

Embodiment 7: The device of any of embodiments 1-6, wherein the article of clothing comprises a belt.

Embodiment 8: The device of any of embodiments 1-7, wherein the reservoir is integrally formed within the housing.

Embodiment 9: The device of any of embodiments 1-8, wherein the reservoir is configured for insertion into the housing.

Embodiment 10: The device of any of embodiments 1-9, wherein the reservoir is configured for attachment to the housing.

Embodiment 11: The device of any of embodiments 1-10, wherein the reservoir comprises a syringe, a vial, or a cartridge.

Embodiment 12: The device of any of embodiments 1-11, wherein the reservoir is pre-filled with the pharmaceutical formulation.

Embodiment 13: The device of any of embodiments 1-12, wherein the dispenser is a dispensing needle in fluid communication with the reservoir and configured for placement into or through a tissue or a cavity of the patient;

Embodiment 14: The device of any of embodiments 1-13, wherein the dispensing needle comprises a multi-beveled tip.

Embodiment 15: The device of any of embodiments 1-14, wherein the pump or spring is configured to actuate delivery of the pharmaceutical formulation from the reservoir through the dispensing needle and into the tissue or a cavity of the patient;

Embodiment 16: The device of any of embodiments 1-15, wherein the pump an injection controller operable to automate delivery of the pharmaceutical formulation by the pump or spring

Embodiment 17: The device of any of embodiments 1-16, wherein the pump is a positive displacement pump.

Embodiment 18: The device of any of embodiments 1-17, wherein the positive displacement pump comprises a rotating piston.

Embodiment 19: The device of any of embodiments 1-18, wherein the positive displacement pump comprises a slidable piston.

Embodiment 20: The device of any of embodiments 1-19, wherein the pump is an osmotic pump.

Embodiment 21: The device of any of embodiments 1-20, wherein the device is configured for wirelessly receiving information from a handheld controller about one or more operations of the device.

Embodiment 22: The device of any of embodiments 1-21, wherein the viscosity reducer is selected from glycylglycine, glycine, a hyaluronidase, sodium citrate, sodium succinate, histidine, TRIS buffer, HEPES buffer, sodium chloride, calcium chloride, magnesium chloride, arginine, lidocaine, benzyl alcohol and/or polysorbate-80, succinic acid, acetic acid, phosphoric acid, tartaric acid, amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, or Solutol HS 15.

Embodiment 23: The device of any of embodiments 1-22, wherein the pharmaceutical formulation further comprises a buffer or excipient so that the nucleic acid is in the form of an alkali metal salt.

Embodiment 24: The device of any of embodiments 1-23, wherein the viscosity reducer is glycine, glycylglycine, sodium citrate, sodium succinate, or sodium chloride.

Embodiment 25: The device of any of embodiments 1-24, wherein the buffer or excipient is sodium citrate, sodium succinate, histidine, TRIS buffer, HEPES buffer, sodium chloride, arginine, lidocaine, benzyl alcohol and/or polysorbate-80.

Embodiment 26: The device of any of embodiments 1-25, wherein the buffer or excipient is sodium citrate at a concentration.

Embodiment 27: The device of any of embodiments 1-26, wherein the viscosity of the pharmaceutical formulation decreases over time.

Embodiment 28: The device of any of embodiments 1-27, wherein the viscosity of the pharmaceutical formulation decreases during storage.

Embodiment 29: The device of any of embodiments 1-28, wherein the viscosity of the pharmaceutical formulation decreases under increasing shear, agitation, temperature, time, and/or pressure.

Embodiment 30: The device of any of embodiments 1-29, wherein said shear increases during administration.

Embodiment 31: The device of any of embodiments 1-30, wherein the shear increases during administration via the dispensing needle.

Embodiment 32: The device of any of embodiments 1-31, wherein the pharmaceutical formulation is formulated for subcutaneous delivery.

Embodiment 33: The device of any of embodiments 1-32, wherein the pharmaceutical formulation demonstrates extended systemic half-life compared to a defibrotide formulation not comprising glycylglycine.

Embodiment 34: The device of any of embodiments 1-33, wherein the pharmaceutical formulation exhibits lower peak-to-trough ratios of plasma concentrations compared to a defibrotide formulation not comprising glycylglycine.

Embodiment 35: The device of any of embodiments 1-34, wherein the pharmaceutical formulation exhibits improved efficacy and/or an improved safety profile compared to a defibrotide formulation not comprising glycylglycine.

Embodiment 36: The device of any of embodiments 1-35, wherein the pharmaceutical formulation is isotonic, hypertonic or thixotropic

Embodiment 37: The device of any of embodiments 1-36, wherein the device is packaged for self-administration by a patient.

Embodiment 38: The device of any of embodiments 1-37, wherein the tissue is subcutaneous tissue.

Embodiment 39: The device of any of embodiments 1-38, wherein the tissue is a muscle of the patient.

Embodiment 40: The device of any of embodiments 1-39, wherein the tissue is dermal tissue.

Embodiment 41: The device of any of embodiments 1-40, wherein the tissue is the peritoneum.

Embodiment 42: The device of any of embodiments 1-40, wherein the tissue is nasal.

Embodiment 43: The device of any of embodiments 1-42, wherein the cavity is the peritoneal cavity of the patient.

Embodiment 44: A patch for delivering a pharmaceutical formulation to a patient comprising an adhesive for removably securing the patch to the patient, wherein the pharmaceutical formulation comprises between about 90 mg/mL to about 400 mg/mL of defibrotide, and a viscosity reducer at a concentration of between about 5 mM and about 350 mM, and wherein the formulation has a viscosity between about 5 and about 70 cP when measured at between 15° C. and 25° C., and an osmolality between about 240 mOsm/kg and about 1000 mOsm/kg.

Embodiment 45: The patch of embodiment 44, wherein the pharmaceutical formulation is contained within a reservoir of the patch.

Embodiment 46: The patch of any of embodiments 44-45, wherein the patch is configured for transdermal delivery.

Embodiment 47: The patch of any of embodiments 44-46, wherein the patch is configured for intradermal delivery.

Embodiment 48: The patch of any of embodiments 44-47, wherein the patch comprises a plurality of dispensing needles.

Embodiment 49: The patch of any of embodiments 44-48, wherein the pharmaceutical formulation is contained within the plurality of dispensing needles.

Embodiment 50: The patch of any of embodiments 44-49, wherein the plurality of dispensing needles comprise a coating, and wherein the coating comprises the pharmaceutical formulation.

Embodiment 51: A device for delivering a pharmaceutical formulation to a patient comprising:

(a) a housing;
(b) a reservoir coupled to the housing for containing the pharmaceutical formulation;
(c) a dispensing needle in fluid communication with the reservoir and configured for placement into or through a tissue or a cavity of the patient;
(d) a releasable stressed spring configured to actuate delivery of the pharmaceutical formulation from the reservoir through the dispensing needle and into the tissue or a cavity of the patient; and
wherein the pharmaceutical formulation comprises between about 90 mg/mL to about 400 mg/mL of defibrotide, and a viscosity reducer at a concentration of between about 5 mM and about 350 mM, and wherein the formulation has a viscosity between about 5 and about 70 cP when measured at between 15° C. and 25° C., and an osmolality between about 240 mOsm/kg and about 1000 mOsm/kg.

Embodiment 52: The device of embodiment 51, wherein the housing is configured to be off-body or removably secured to the patient

Embodiment 53: A device for delivering a pharmaceutical formulation to a patient comprising:

(a) a housing;
(b) a reservoir coupled to the housing for containing the pharmaceutical formulation;
(c) a dispensing tip in fluid communication with the reservoir and configured for placement into or through a tissue or a cavity of the patient;
(d) a plunger configured to actuate delivery of the pharmaceutical formulation from the reservoir through the dispensing tip and into the tissue or a cavity of the patient; and, wherein the pharmaceutical formulation comprises between about 900 mg/mL to about 400 mg/mL of defibrotide, and a viscosity reducer at a concentration of between about 5 mM and about 350 mM, and wherein the formulation has a viscosity between about 5 and about 70 cP when measured at between 15° C. and 25° C., and an osmolality between about 240 mOsm/kg and about 1000 mOsm/kg.

Embodiment 54: The device of embodiment 53, wherein the housing is configured to be off-body or removably secured to the patient.

Embodiment 55: The device of embodiment 53 or 54, wherein the device is configured for manual application of a force to actuate delivery of the formulation.

Embodiment 56: The device of any of embodiments 53-55, wherein the viscosity reducer is selected from glycylglycine, glycine, a hyaluronidase, sodium citrate, sodium succinate, histidine, TRIS buffer, HEPES buffer, sodium chloride, arginine, lidocaine, benzyl alcohol and/or polysorbate-80, succinic acid, acetic acid, phosphoric acid, tartaric acid, amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, or Solutol HS 15.

Embodiment 57: The device of any of embodiments 53-56, wherein the pharmaceutical formulation further comprises a buffer or excipient so that the nucleic acid is in the form of an alkali metal salt.

Embodiment 58: The device of any of embodiments 53-57, wherein the buffer or excipient is sodium citrate, sodium succinate, histidine, TRIS buffer, HEPES buffer, sodium chloride, arginine, lidocaine, benzyl alcohol and/or polysorbate-80.

Embodiment 59: The device of any of embodiments 53-58, wherein the buffer or excipient is sodium citrate, sodium succinate, or sodium chloride at a concentration of less than about 80 mM sodium salt.

Embodiment 60: The device of any of embodiments 53-59, wherein the buffer or excipient is sodium citrate at a concentration of between about 20 mM and about 34 mM.

Embodiment 61: The device of any of embodiments 53-60, wherein the viscosity of the pharmaceutical formulation decreases over time.

Embodiment 62: The device of any of embodiments 53-61, wherein the viscosity of the pharmaceutical formulation decreases during storage.

Embodiment 63: The device of any of embodiments 53-62, wherein the viscosity of the pharmaceutical formulation decreases under increasing shear, agitation, temperature, time, and/or pressure.

Embodiment 64: The device of any of embodiments 53-63, wherein said shear increases during administration.

Embodiment 65: The device of any of embodiments 53-64, wherein the shear increases during administration via the dispensing needle.

Embodiment 66: The device of any of embodiments 53-65, wherein the pharmaceutical formulation is formulated for subcutaneous, intramuscular, intradermal, intraocular, intraperitoneal, intranasal, or inhaled delivery.

Embodiment 67: The device of any of embodiments 53-66, wherein the pharmaceutical formulation demonstrates extended systemic half-life compared to a defibrotide formulation not comprising a viscosity reducer.

Embodiment 68: The device of any of embodiments 53-67, wherein the pharmaceutical formulation exhibits lower peak-to-trough ratios of plasma concentrations compared to a defibrotide formulation administered intravenously.

Embodiment 69: The device of any of embodiments 53-68, wherein the pharmaceutical formulation exhibits improved efficacy and/or an improved safety profile compared to a defibrotide formulation not comprising a viscosity reducer.

Embodiment 70: The device of any of embodiments 53-69, wherein the pharmaceutical formulation is isotonic, hypertonic or thixotropic.

Embodiment 71: The device of any of embodiments 53-70, wherein the device is packaged for self-administration by a patient.

Embodiment 72: The device of any of embodiments 53-71, wherein the cavity is the nasal or oral cavity.

Embodiment 73: The device of any of embodiments 53-72, wherein the tissue is a muscle of the patient.

Embodiment 74: The device of any of embodiments 53-73, wherein the tissue is dermal tissue.

Embodiment 75: The device of any of embodiments 53-74, wherein the tissue is the peritoneum.

Embodiment 76: The device of any of embodiments 53-75, wherein the cavity is the peritoneal cavity of the patient.

Embodiment 77. A method for defibrotide administration to a patient using any one of the devices of embodiments 1-76.

Embodiment 78: The method of embodiment 77, wherein the pharmaceutical formulation is administered to treat a disease or a condition selected from the group consisting of thrombosis, Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury (IRI, including Kidney IRI), treatment and prevention of cytokine release syndrome (CRS) or Chimeric Antigen Receptor (CAR)-T Cell Related Encephalopathy Syndrome (CRES) or CAR-T neurotoxicity, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies, and Solid Tumors.

Embodiment 79: The method of any of embodiments 77-78, wherein the pharmaceutical formulation is administered at a dosing regimen that provides improved patient quality of life by requiring a reduced administration volume and/or allowing less-frequent administration and/or a shorter duration of administration.

Embodiment 80: The method of any of embodiments 77-79, wherein the pharmaceutical formulation is administered subcutaneously to the patient.

Embodiment 81: The method of any of embodiments 77-80, wherein the pharmaceutical formulation is administered intramuscularly to the patient.

Embodiment 82: The method of any of embodiments 77-81, wherein the pharmaceutical formulation is administered intradermally to the patient.

Embodiment 83: The method of any of embodiments 77-82, wherein the pharmaceutical formulation is administered transdermally to the patient.

Embodiment 84: The method of any of embodiments 77-83, wherein the pharmaceutical formulation is administered nasally to the patient.

Embodiment 85: The method of any of embodiments 77-84, wherein the pharmaceutical formulation is administered through or into the peritoneum of the patient.

Embodiment 86: The method of any of embodiments 77-84, wherein the pharmaceutical formulation is administered in an outpatient or at-home setting.

### 6. EXAMPLES

### 6.1 Example 1—Identification of Limiting Solution Attributes

In order to develop low viscosity formulations of a nucleic acid, it is important to identify key physicochemical properties of the solution that may limit formulation. To investigate this, a number of solutions ranging in defibrotide concentrations of up to approximately 300 mg/mL were generated using two different formulations and the solution properties of each were characterized as a function of defibrotide concentration. The visual appearance, solubility, viscosity, osmolality, polymer structure in solution (far- and near-UV Circular Dichroism), thermal properties (Differential Scanning Calorimetry), and molecular weight/aggregation (DLS, FTIR, and SEC-MALS) were some of the properties analyzed for the various defibrotide solutions.

Sample Preparation: defibrotide was formulated in 34 mM sodium citrate, pH 7.3 or 34 mM glycylglycine (“Gly-Gly”), pH 7.5 by centrifugal concentration or by dissolving defibrotide API at 80, 150, 200, 250, and 300 mg/mL. Product concentration was typically measured by Ultraviolet-Visible Spectroscopy. Defibrotide samples were diluted gravimetrically in triplicate to a target concentration of 0.1 mg/mL in their respective formulation buffers using 35 μL of sample. A₂₆₀ and A₃₂₀ were measured using a cuvette path length of 0.2 cm. A₂₆₀ values were corrected for light scattering at 320 nm and the concentration was determined using an extinction coefficient of 22.2 mL*mg⁻¹cm⁻¹. A sample density of 1.08 g/mL and a diluent density of 1.0 g/mL were used to correct the mass of the sample when determining the dilution factor. The physiochemical properties of each solution were analyzed as a function of concentration by the following methods:

Visual Appearance & Turbidity: digital color matching was performed by a Core Module 3 (“CM3”) robot. The color of defibrotide samples was also evaluated using the European Pharmacopeia (EP) color matching analysis using seven EP color standards, BY1-BY7, with BY1 being the most intensely colored standard. The analysis was conducted under a light box with a white background (Eisai Machinery Observation Lamp Model MIH-DX, Fisher light Meter Model 06-662-63). The color evaluations for the Gly-Gly and citrate formulations were not significantly different; all were clear slightly yellow, or brown—yellow solutions with the intensity of coloration being more pronounced than the standard. In addition, no visible particles were detected (particle sizes of ≥80 μm were evaluated). Turbidity was also measured against seven turbidity standards. The color of all formulations when compared to the EP color standards was BY4 at the initial time point as well as after one month of storage at 25±2° C./60±5% RH showing that all formulations were stable for up to at least three months at 25±2° C./60±5% RH.

Solubility: the solubility of defibrotide in solution was evaluated via polyethylene glycol (PEG) precipitation using the CM3 robot for analysis. Throughout the studies, a miniscule amount of precipitation was observed even in the presence of a high quantity PEG, thus indicating high solution solubility of the product.

Solution Viscosity: the solution viscosity of defibrotide samples were analyzed at approximately 80, 150, 200, 250, and 300 mg/mL concentrations. Typically a Brookfield DV-III Ultra Programmable Rheometer was used to measure the viscosity. The samples were analyzed neat at 22° C. using approximately 550 μL. The viscosity of Defitelio® was 3.9 cP with no dependence on shear rate. The results suggested that Defitelio® displayed Newtonian fluid behavior. Defibrotide formulations of the invention formulated at 300 mg/mL in citrate and Gly-Gly buffers demonstrated that the viscosity was dependent of shear rate and product concentration. The viscosity of the formulated defibrotide appeared to increase exponentially as a function of the product concentration. The viscosity of defibrotide in 34 mM Gly-Gly, pH 7.5 as a function of product concentration was significantly lower compared to defibrotide in 34 mM sodium citrate and pH 7.3 demonstrating the ability of Gly-Gly to improve the solution properties of defibrotide in the HCLFs of the invention.

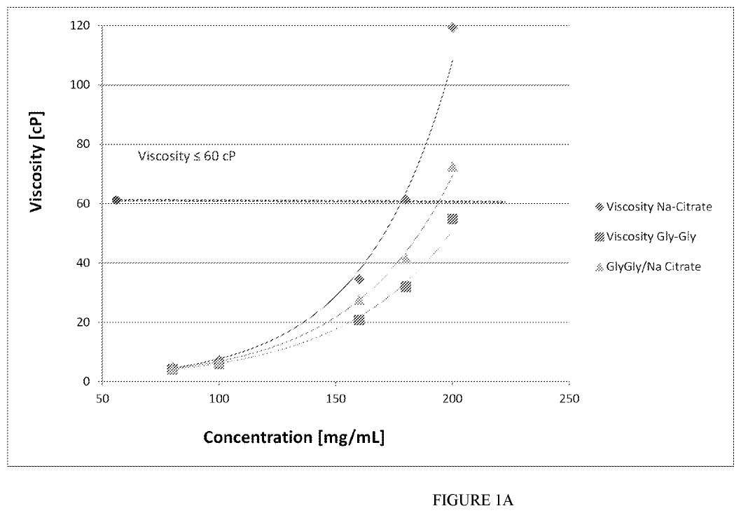

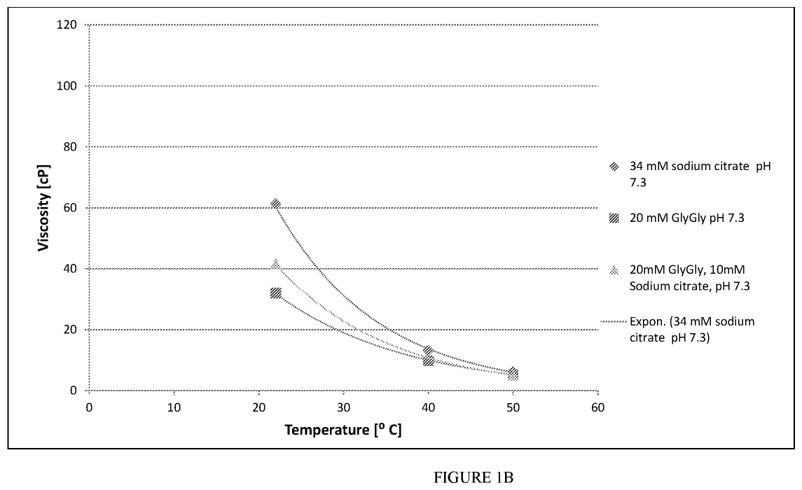

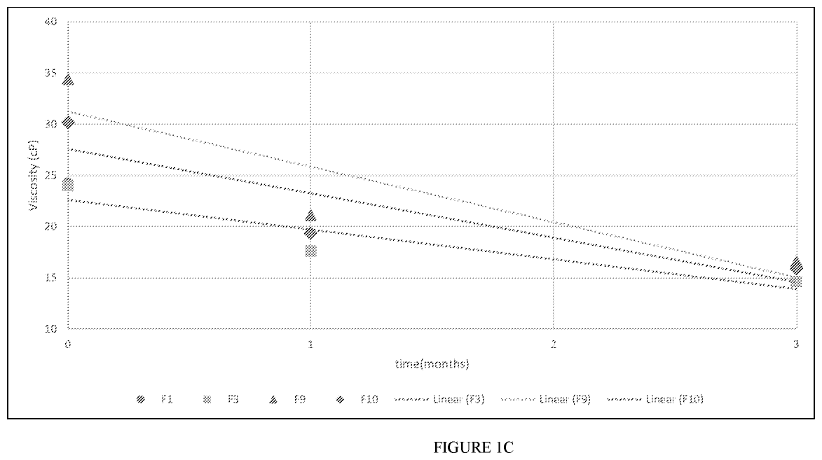

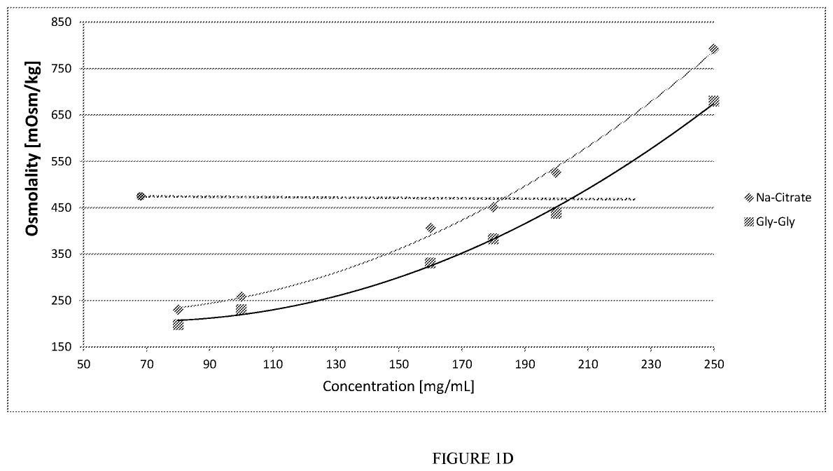

Osmolality: The osmolality was measured by at least two different methods and results are reported in the Figures throughout (see, for example, FIG. 1D). Typically, a Vapro Vapor Pressure Osmometer was used for one measurement. The osmolality of defibrotide in 34 mM Gly-Gly, pH 7.5 was lower compared to defibrotide in 34 mM sodium citrate and pH 7.3 demonstrating the ability of Gly-Gly to improve the solution properties of defibrotide in the HCLFs of the invention.

Far and Near-Ultraviolet Circular Dichroism: the secondary and tertiary structure of defibrotide formulations in solution, as a function of product concentration, was assessed by circular dichroism and analyzed on a Jasco J-810 Spectropolarimeter.

Free Nucleic Bases Analysis: Samples were quantitatively prepared at 1.6 mg/mL with mobile phase (50 mM CH₃COONH₄, pH 5.0) and analyzed by RP-HPLC using a detection wavelength of 254 nm. A Synergi Fusion 4 μm-RP 80 Å column was used to separate the nucleic base using a flow rate of 1 mL/min. Defitelio® was used as a reference and was prepared at 1.6 mg/mL in mobile phase.

Fourier Transform Infrared Spectroscopy: FTIR analysis was performed using standard techniques to evaluate the structure of HCLFs of defibrotide. The FTIR analysis demonstrated that the two defibrotide formulations (citrate and Gly-Gly) at 300 mg/mL displayed a similar profile when compared to Defitelio®.

Differential Scanning Calorimetry: the thermal properties in solution of defibrotide were measured by differential scanning calorimetry using standard techniques. The results suggest that concentration and/or buffer matrix, including Gly-Gly, can influence the thermal properties of defibrotide.

Size Distribution: measured for each formulation as a function of the product concentration in order to account for the molecular weight of contributing structures to the molecular weighted average. The polydispersity index (Mw/Mn) was used to measure the heterogeneity of the formulations and, based on the results, the samples were concluded to be polydispersed. The results showed that defibrotide formulated at 300 mg/mL in citrate and Gly-Gly is comparable to Defitelio®.

Overall, the results using the above methods indicate that the solution osmolality and viscosity are important formulation attributes playing a critical role in limiting how high product concentration can be achieved that is well tolerated. These attributes in Gly-Gly containing formulations demonstrated notable solution properties improvements which also correlate with thermal attributes in solution (ΔH, Tm).

The graph in FIG. 1A shows the viscosity of formulations made using increasing defibrotide concentrations in the presence of sodium citrate, Gly-Gly or a mixture of the two. The results show that the viscosity of defibrotide formulations is strongly dependent on its concentration, and a 200 mg/mL solution has roughly 10-fold higher viscosity as compared to the 80 mg/mL solution.

The graph is FIG. 1B shows the viscosity as a function of temperature in three different formulations comprising either sodium citrate, Gly-Gly or a mixture of the two.

The graph in FIG. 1C shows the viscosity decrease over the course of time in these selected formulations: 20 mM GlyGly (blue circles; overlapped by the orange squares), 20 mM GlyGly and 34 mM sodium citrate (orange squares), 20 mM GlyGly and 100 mM sodium succinate (blue triangles) and 20 mM GlyGly and 20 mM sodium chloride (red diamonds). GlyGly containing formulations show the lowest viscosity for a given time point.

The graph in FIG. 1D shows the osmolality of formulations made using increasing defibrotide concentrations in the presence of Gly-Gly or sodium citrate buffers.

### 6.2 Example 2—Effect of Buffers on Formulation Properties

### 6.2.1 Example 2.1—Effect of Buffers and Excipients on Viscosity & Osmolality

Increasing the defibrotide concentration was shown in Example 1 to increase both viscosity and osmolality. It is important for pharmaceutical preparations for oral or parenteral administration to be of low-viscosity and/or hypertonic, isotonic or thixotropic. In order to identify buffers or excipients that may lower the viscosity and/or osmolality of defibrotide formulations, a wide-panel screening of various buffers and excipients (including GRAS excipients) was performed using a 200 mg/mL defibrotide formulation.

Test formulations were prepared to target 200 mg/mL as shown in Table 1 below.

TABLE 1
Defibrotide Formulations using Various Buffers and Excipients

| | Defibrotide | | | | |
|---|---|---|---|---|---|
| | Con- | | Average | Vis- | Shear |
| | centration | Osmolality | Viscosity | cosity | Rate |
| Formulation | (mg/mL) | (mmol/kg) | (cP) | (cP) | (s⁻¹) |
| Defitelio DP, 34 | 80 | Not | 4.2 | 4.21 | 113 |
| mM Sodium | | Tested | | 4.24 | 225 |
| Citrate, pH 7.3 | | | | 4.23 | 338 |
| | | | | 4.28 | 525 |
| 34 mM Sodium | 200 | 438 | 29.6 | 29.6 | 15 |
| Citrate, pH 7.3 | | | | 29.4 | 30 |
| | | | | 29.8 | 48.8 |
| | | | | 29.7 | 71.3 |
| 34 mM Sodium | | 544 | 58 | 61.9 | 7.5 |
| Citrate, pH 6.5 | | | | 57.9 | 22.5 |
| | | | | 56.5 | 30 |
| | | | | 55.5 | 37.5 |
| 34 mM Sodium | | 636 | 51.9 | 51.8 | 7.5 |
| Citrate, 100 mM | | | | 51.8 | 22.5 |
| NaCl, pH 7.3 | | | | 52 | 30 |
| | | | | 51.9 | 37.5 |
| 34 mM Sodium | | 643 | 46.1 | 46.6 | 3.45 |
| Citrate, 100 mM | | | | 45.5 | 15 |
| Arginine, pH 7.3 | | | | 46 | 30 |
| | | | | 46.2 | 45 |
| 34 mM Sodium | | 542 | 53.6 | 52.9 | 11.3 |
| Citrate, 0.1% | | | | 53.7 | 22.5 |
| PS-80, pH 7.3 | | | | 53.9 | 33.8 |
| | | | | 53.8 | 38.3 |
| 34 mM Sodium | | 994 | 38.3 | 38.3 | 15 |
| Citrate, 250 mM | | | | 38.2 | 30 |
| Lidocaine HCl, | | | | 38.4 | 37.5 |
| pH 7.0 | | | | 38.4 | 52.5 |
| 34 mM Sodium | | 435 | 46.4 | 46.1 | 15 |
| Citrate, pH 8.0 | | | | 46.3 | 22.4 |
| | | | | 46.4 | 30 |
| | | | | 46.6 | 45 |
| 34 mM Gly-Gly, | | 566 | 38.5 | 38.6 | 15 |
| 100 mM NaCl, | | | | 38.4 | 30 |
| pH 7.5 | | | | 38.5 | 37.5 |
| | | | | 38.6 | 54 |
| 34 mM Gly-Gly, | 200 | 560 | 39.7 | 39.4 | 15 |
| 100 mM | | | | 39.5 | 30 |
| Arginine, pH 7.5 | | | | 39.7 | 45 |
| | | | | 40.3 | 52.5 |
| 34 mM Gly-Gly, | | 359 | 38.2 | 37.9 | 15 |
| 0.1% PS-80, | | | | 38.2 | 30 |
| pH 7.5 | | | | 38.2 | 45 |
| | | | | 38.4 | 54 |
| 34 mM Gly-Gly, | | 950 | 34.1 | 34 | 15 |
| 250 mM | | | | 34.1 | 37.5 |
| Lidocaine HCl, | | | | 34.2 | 48.8 |
| pH 7.0 | | | | 34.6 | 60 |
| 34 mM Gly-Gly, | | 323 | 27.7 | 27.9 | 16.5 |
| pH 7.5 | | | | 27.7 | 31.5 |
| | | | | 27.6 | 51 |
| | | | | 27.7 | 75 |
| 34 mM Gly-Gly, | | 370 | 36.9 | 36.5 | 15 |
| pH 8.0 | | | | 37 | 33.8 |
| | | | | 37 | 45 |
| | | | | 37.2 | 56.3 |
| 34 mM Gly-Gly, | | 375 | 34 | 33.7 | 15 |
| pH 8.5 | | | | 33.7 | 37.5 |
| | | | | 34.1 | 48.8 |
| | | | | 34.3 | 60.8 |
| 34 mM Tris, pH | | 394 | 39.4 | 39.1 | 15 |
| 7.5 | | | | 39.2 | 30 |
| | | | | 39.4 | 45 |
| | | | | 39.8 | 52.5 |
| 34 mM HEPES, | | 379 | 43.9 | 43.8 | 15 |
| pH 7.5 | | | | 43.8 | 30 |
| | | | | 43.7 | 37.5 |
| | | | | 44.2 | 46.5 |
| 34 mM His, pH | | 364 | 37 | 36.9 | 15 |
| 7.3 | | | | 36.9 | 30 |
| | | | | 37 | 45 |
| | | | | 37.3 | 56.3 |

TABLE 2
Solution viscosity and osmolality of defibrotide in Gly-Gly containing buffer
in comparison to sodium citrate as a function of product concentration

| | | | | | Mean | Shear | |
|---|---|---|---|---|---|---|---|
| | Concentration | Temp. | | Viscosity | Viscosity | Rate | Osmolality |
| Formulation | (mg/mL) | (° C.) | | (cP) | (cP) | (s⁻¹) | Mmol/kg |
| 34 mM | 80 | 2-8° | C. | 11.5 | 11.6 | 37.5 | |
| Sodium | | | | 11.6 | | 75.0 | |
| Citrate | | | | 11.6 | | 113 | |
| pH 7.3 | | | | 11.8 | | 150 | |
| | | 22° | C. | 4.9 | 4.9 | 60.0 | 230 |
| | | | | 4.9 | | 150.0 | |
| | | | | 4.9 | | 263.0 | |
| | | | | 4.9 | | 413.0 | |
| | | 40° | C. | 2.3 | 2.3 | 113.0 | |
| | | | | 2.3 | | 300.0 | |
| | | | | 2.3 | | 450.0 | |
| | | | | 2.4 | | 900.0 | |
| | | 50° | C. | 1.56 | 1.6 | 225 | |
| | | | | 1.58 | | 450 | |
| | | | | 1.58 | | 900 | |
| | | | | 1.60 | | 1200 | |
| | 100 | 2-8° | C. | 20.2 | 20.7 | 22.5 | |
| | | | | 20.5 | | 37.5 | |
| | | | | 20.9 | | 75.0 | |
| | | | | 21.2 | | 97.5 | |
| | | 22° | C. | 7.5 | 7.3 | 37.5 | 259 |
| | | | | 7.2 | | 75.0 | |
| | | | | 7.2 | | 150 | |
| | | | | 7.2 | | 300 | |
| | | 40° | C. | 3.0 | 3.0 | 113 | |
| | | | | 3.0 | | 263 | |
| | | | | 3.0 | | 413 | |
| | | | | 3.1 | | 675 | |
| | | 50° | C. | 1.96 | 2.0 | 150 | |
| | | | | 1.95 | | 300 | |
| | | | | 1.98 | | 675 | |
| | | | | 1.99 | | 975 | |
| | 160 | 2-8° | C. | 628.3 | 655.5 | 0.750 | |
| | | | | 648.1 | | 1.50 | |
| | | | | 669.9 | | 2.25 | |
| | | | | 675.5 | | 3.00 | |
| | | 22° | C. | 34.6 | 34.5 | 15.0 | 407 |
| | | | | 34.4 | | 22.5 | |
| | | | | 34.4 | | 37.5 | |
| | | | | 34.4 | | 60.0 | |
| | | 40° | C. | 8.2 | 8.6 | 37.5 | |
| | | | | 8.3 | | 75.0 | |
| | | | | 8.4 | | 150 | |
| | | | | 9.4 | | 225 | |
| | | 50° | C. | 4.26 | 4.4 | 75.0 | |
| | | | | 4.31 | | 156 | |
| | | | | 4.39 | | 300 | |
| | | | | 4.50 | | 450 | |
| | 180 | 2-8° | C. | N/A | <600 | N/A | |
| | | | | N/A | | N/A | |
| | | | | N/A | | N/A | |
| | | | | N/A | | N/A | |
| | | 22° | C. | 61.8 | 61.3 | 4.50 | 451 |
| | | | | 60.7 | | 7.50 | |
| | | | | 61.4 | | 15.0 | |
| | | | | 61.3 | | 30.0 | |
| | | 40° | C. | 11.5 | 13.1 | 37.5 | |
| | | | | 12.6 | | 75.0 | |
| | | | | 13.5 | | 113.0 | |
| | | | | 14.8 | | 150.0 | |
| | | 50° | C. | 5.85 | 6.2 | 75.0 | |
| | | | | 6.01 | | 150 | |
| | | | | 6.31 | | 225 | |
| | | | | 6.47 | | 300 | |
| | 200 | 2-8° | C. | N/A | <600 | N/A | |
| | | | | N/A | | N/A | |
| | | | | N/A | | N/A | |
| | | | | N/A | | N/A | |
| | | 22° | C. | 117.1 | 119.4 | 3.75 | 525 |
| | | | | 119.2 | | 6.00 | |
| | | | | 119.9 | | 11.3 | |
| | | | | 121.5 | | 15.0 | |
| | | 40° | C. | 17.3 | 18.3 | 22.5 | |
| | | | | 17.9 | | 52.5 | |
| | | | | 18.4 | | 75.0 | |
| | | | | 19.5 | | 113.0 | |
| | | 50° | C. | 7.26 | 7.5 | 75.0 | |
| | | | | 7.34 | | 150 | |
| | | | | 7.42 | | 225 | |
| | | | | 7.88 | | 263 | |
| | 250 | 2-8° | C. | N/A | <600 | N/A | |
| | | | | N/A | | N/A | |
| | | | | N/A | | N/A | |
| | | | | N/A | | N/A | |
| | | 22° | C. | N/A | <600 | N/A | 792 |
| | | | | N/A | | N/A | |
| | | | | N/A | | N/A | |
| | | | | N/A | | N/A | |
| | | 40° | C. | N/A | <600 | N/A | |
| | | | | N/A | | N/A | |
| | | | | N/A | | N/A | |
| | | | | N/A | | N/A | |
| | | 50° | C. | 31.60 | 28.5 | 7.50 | |
| | | | | 27.60 | | 37.5 | |
| | | | | 27.20 | | 52.5 | |
| | | | | 27.70 | | 75.0 | |

| | | | | | Mean | Shear | |
|---|---|---|---|---|---|---|---|
| | Concentration | Temp. | | Viscosity | Viscosity | Rate | Osmolality |
| Formulation | (mg/mL) | (° C.) | | (cP) | (cP) | (s⁻¹) | mmol/kg |
| 20 mM | 80 | 2-8° | C. | 8.95 | 9.1 | 37.5 | |
| Gly-Gly | | | | 9.10 | | 75.0 | |
| pH 7.3 | | | | 9.15 | | 150 | |
| | | | | 9.18 | | 188 | |
| | | 22° | C. | 4.1 | 4.2 | 75.0 | 198 |
| | | | | 4.1 | | 150.0 | |
| | | | | 4.2 | | 225.0 | |
| | | | | 4.2 | | 413.0 | |
| | | 40° | C. | 2.3 | 2.4 | 113.0 | |
| | | | | 2.3 | | 300.0 | |
| | | | | 2.4 | | 450.0 | |
| | | | | 2.4 | | 825.0 | |
| | | 50° | C. | 1.61 | 1.6 | 225 | |
| | | | | 1.61 | | 450 | |
| | | | | 1.62 | | 900 | |
| | | | | 1.64 | | 1200 | |
| | 100 | 2-8° | C. | 14.2 | 14.3 | 37.5 | |
| | | | | 14.3 | | 75.0 | |
| | | | | 14.4 | | 113.0 | |
| | | | | 14.4 | | 150.0 | |
| | | 22° | C. | 6.0 | 6.0 | 75.0 | 231 |
| | | | | 6.0 | | 113.0 | |
| | | | | 6.0 | | 188.0 | |
| | | | | 6.0 | | 300.0 | |
| | | 40° | C. | 2.9 | 2.9 | 113.0 | |
| | | | | 2.9 | | 263.0 | |
| | | | | 2.9 | | 413.0 | |
| | | | | 2.9 | | 675.0 | |
| | | 50° | C. | 2.02 | 2.1 | 156 | |
| | | | | 2.05 | | 300 | |
| | | | | 2.08 | | 675 | |
| | | | | 2.11 | | 900 | |
| | 160 | 2-8° | C. | 88.1 | 88.0 | 6.00 | |
| | | | | 86.7 | | 7.50 | |
| | | | | 88.5 | | 11.3 | |
| | | | | 88.6 | | 22.5 | |
| | | 22° | C. | 20.8 | 20.8 | 15.0 | 331 |
| | | | | 20.7 | | 37.5 | |
| | | | | 20.8 | | 75.0 | |
| | | | | 20.8 | | 97.5 | |
| | | 40° | C. | 7.2 | 7.3 | 37.5 | |
| | | | | 7.3 | | 75.0 | |
| | | | | 7.3 | | 188.0 | |
| | | | | 7.5 | | 263 | |
| | | 50° | C. | 4.10 | 4.2 | 150 | |
| | | | | 4.09 | | 300 | |
| | | | | 4.18 | | 450 | |
| | | | | 4.27 | | 488 | |
| | 180 | 2-8° | C. | N/A | <600 | N/A | |
| | | | | N/A | | N/A | |
| | | | | N/A | | N/A | |
| | | | | N/A | | N/A | |
| | | 22° | C. | 31.6 | 32.0 | 7.50 | 383 |
| | | | | 32.2 | | 15.0 | |
| | | | | 32.0 | | 37.5 | |
| | | | | 32.2 | | 60.0 | |
| | | 40° | C. | 9.6 | 9.8 | 37.5 | |
| | | | | 9.7 | | 75.0 | |
| | | | | 9.9 | | 113.0 | |
| | | | | 10.2 | | 188.0 | |
| | | 50° | C. | 5.24 | 5.3 | 75.0 | |
| | | | | 5.26 | | 150 | |
| | | | | 5.33 | | 300 | |
| | | | | 5.43 | | 375 | |
| | 200 | 2-8° | C. | N/A | <600 | N/A | |
| | | | | N/A | | N/A | |
| | | | | N/A | | N/A | |
| | | | | N/A | | N/A | |
| | | 22° | C. | 54.0 | 54.9 | 7.50 | 438 |
| | | | | 55.4 | | 11.3 | |
| | | | | 55.0 | | 18.8 | |
| | | | | 55.0 | | 33.8 | |
| | | 40° | C. | 14.0 | 14.2 | 22.5 | |
| | | | | 14.2 | | 52.5 | |
| | | | | 14.2 | | 90.0 | |
| | | | | 14.3 | | 127.0 | |
| | | 50° | C. | 7.17 | 7.4 | 75.0 | |
| | | | | 7.31 | | 150 | |
| | | | | 7.43 | | 22.5 | |
| | | | | 7.56 | | 263 | |
| | 250 | 2-8° | C. | N/A | <600 | N/A | |
| | | | | N/A | | N/A | |
| | | | | N/A | | N/A | |
| | | | | N/A | | N/A | |
| | | 22° | C. | N/A | <600 | N/A | 680 |
| | | | | N/A | | N/A | |
| | | | | N/A | | N/A | |
| | | | | N/A | | N/A | |
| | | 40° | C. | 46.00 | 44.5 | 15.0 | |
| | | | | 44.40 | | 30.0 | |
| | | | | 44.00 | | 37.5 | |
| | | | | 43.50 | | 41.3 | |
| | | 50° | C. | 23.00 | 22.6 | 37.5 | |
| | | | | 22.00 | | 52.5 | |
| | | | | 22.40 | | 75.0 | |
| | | | | 22.80 | | 90.0 | |

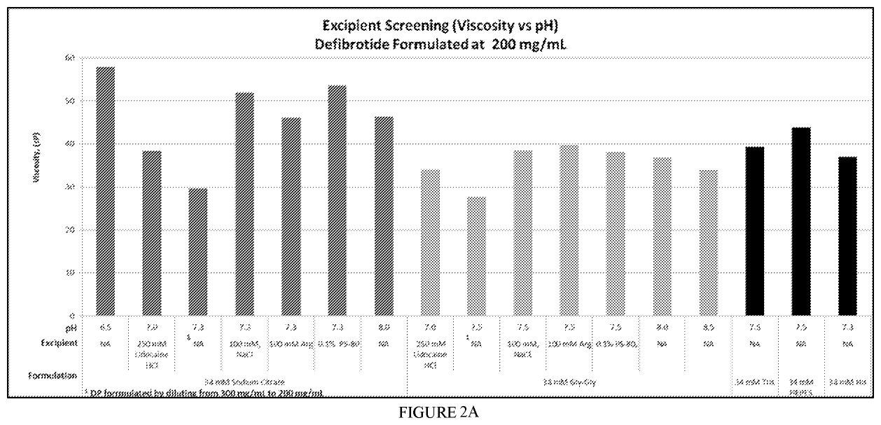

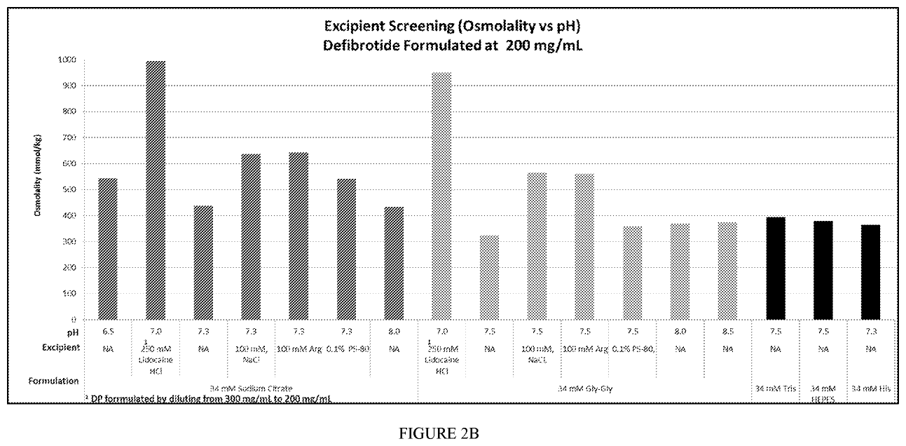

As shown in Tables 1 and 2 as well as the graphs in FIGS. 1B, 1C, and 2A, the HCLF formulations containing Gly-Gly had significantly lower viscosity overall compared to other formulations and when tested under different pH, product concentration, and temperature conditions (see FIG. 2A) as compared to the citrate buffer. Notably, the TRIS and histidine buffers also had lower viscosity (less than 40 cP) at 200 mg/mL defibrotide. For nearly all of the formulations in Gly-Gly, the viscosity was up to 50% lower compared to the citrate buffer for given product concentration and ambient temperature conditions. Ambient temperatures may change depending on the region and therefore, viscosity is preferably measured between about 15° C. to 30° C.; however, it may be slightly higher or lower given different weather conditions. For example, one preferred formulation containing 180 mg/mL of defibrotide (having a mean molecular weight of 14 kDA), and containing 20 mM Gly-Gly and 34 mM sodium citrate pH 7.0, had a viscosity of 12 cP when measured at 25° C. Other formulations had a viscosity of 27 cp when defibrotide having a mean molecular weight of 17 kDa was used under the same conditions. Out of the excipients screened, only lidocaine showed a potential for further reduction of viscosity; however, it increased osmolality >900 mOsm/kg (see FIG. 2B) and therefore was not considered practical for further investigation. Gly-gly buffer showing the lowest viscosity overall was identified as a preferred buffer for 200 mg/mL HCLF defibrotide formulations.

### 6.2.2 Example 2.2—Effect of Different Sodium Ion Sources on Buffering Capacity and Stability During Storage

In order to compare the buffering capacity of various buffer solutions in high concentration low-viscosity defibrotide (“DF”) formulations containing the Gly-Gly buffer system, sixteen different buffers, utilizing three different sodium ion sources were used to evaluate the stability, impurity profile, and solution properties of the DF formulations. A summary of these formulations is shown in Table 3.

TABLE 3
Summary of Defibrotide Formulations

| | | | Sodium | Sodium | |
|---|---|---|---|---|---|
| Formulation | Defibrotide | Gly-Gly | Citrate | Succinate | NaCl |
| Code | (mg/mL) | (mM) | (mM) | (mM) | (mM) |
| F1 | 180 | 20 | — | — | — |
| F2 | 180 | 20 | 20 | — | — |
| F3 | 180 | 20 | 34 | — | — |
| F4 | 180 | 20 | 80 | — | — |
| F5 | 180 | 20 | 100 | — | — |
| F6 | 180 | 20 | — | 20 | — |
| F7 | 180 | 20 | — | 34 | — |
| F8 | 180 | 20 | — | 80 | — |
| F9 | 180 | 20 | — | 100 | — |
| F10 | 180 | 20 | — | — | 20 |
| F11 | 180 | 20 | — | — | 34 |
| F12 | 180 | 20 | — | — | 80 |
| F13 | 180 | 20 | — | — | 100 |
| F14 | 180 | 20 | 40 | — | 40 |
| F15 | 160 | 20 | 40 | — | 40 |
| F16 | 140 | 20 | 40 | — | 40 |

Based on the appearance, color, and clarity results, defibrotide formulated in the Gly-Gly buffers were stable following storage at 25±2° C./60±5% RH for up to at least three months.

UV and pH analysis showed that the defibrotide concentration remained constant for all formulations when stored at 25±2° C./60±5% RH for up to three months.

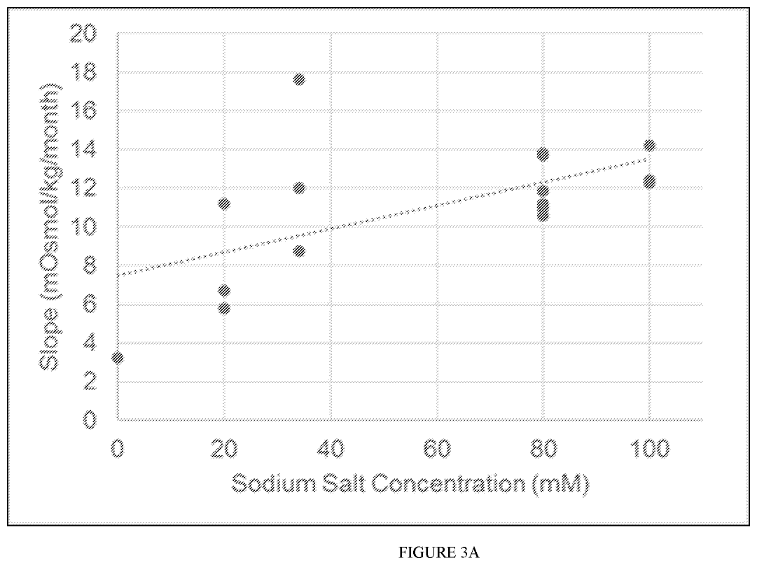

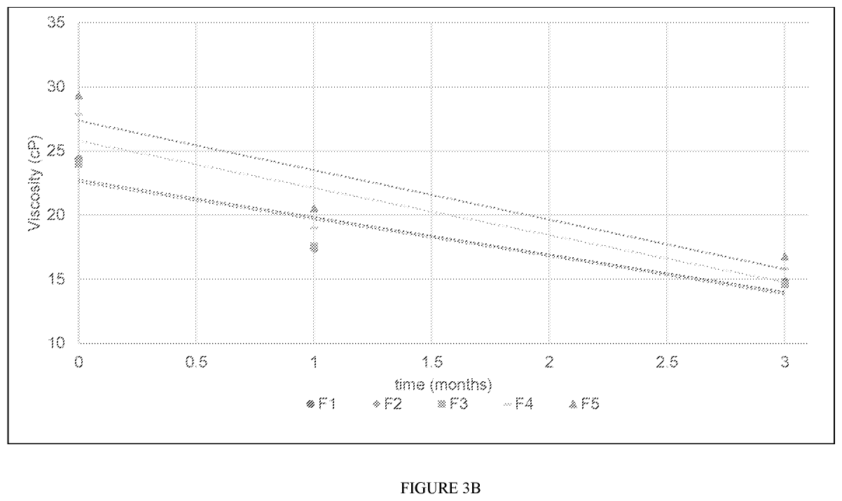

The viscosity of all formulations decreased as a function of time (see for example, FIG. 3B). The viscosity at the initial time point were within the range of 23.8 cP-34.4 cP for all formulations at 180 mg/mL and decreased by up to approximately 52% after storage at 25±2° C./60±5% RH for three months. The formulations F1-F5 as listed in FIG. 3B are described in Table 3 above.

A small osmolality increase trend was observed as a function of storage time that correlated with sodium salt concentration. As salt concentration was increased, the change in increase in osmolality was greater (see FIG. 3A). Formulations with less than 80 mM total sodium salt had the lowest change in osmolality over time and were under 500 mOsm/kg.

The stability indicating FNB assay demonstrated that total impurities and free nucleic bases increased slightly after storage for one month. Overall, formulations F2 and F3 had the lowest amount of free nucleic bases.

Size Exclusion Chromatograph (SEC)-Multi-Angle Light Scattering (MALS) analysis was performed to determine the size distribution and molecular weight of defibrotide as a function of the product concentration. DF formulations and API reference material were diluted to 4 mg/mL in SEC mobile phase in a glass screw cap tube (10 mL). The solution was maintained at room temperature for one hour without stirring. Subsequently, the sample solution was heated to approximately 100° C. (boiling water) and maintained at this temperature for 15 minutes. Finally, the sample solution was cooled using water and ice for five minutes. After stabilization at room temperature (about 15 minutes), the samples were filtered with a 0.20 μm SFCA syringe filter. The sample solution was analyzed by SEC-MALS within one hour of preparation. Reference material was prepared from defibrotide API at 4 mg/mL in mobile phase. The analysis indicated that all formulations have similar sizes and polydispersity

Based on these combined results 180 mg/mL defibrotide in 20 mM Gly-Gly and less than 80 mM sodium salt (and preferably 20-34 mM sodium citrate) are preferred buffer combinations for HCLF formulations.

### 6.3 Example 3—Viscosity Change Over Time and as a Function of Temperature

It is important for pharmaceutical products to maintain their integrity over time to allow for a suitable shelf-life. The viscosity of 200 mg/mL defibrotide formulations using Gly-Gly buffer were therefore measured as a function of both time and temperature.

Samples were prepared as described above.

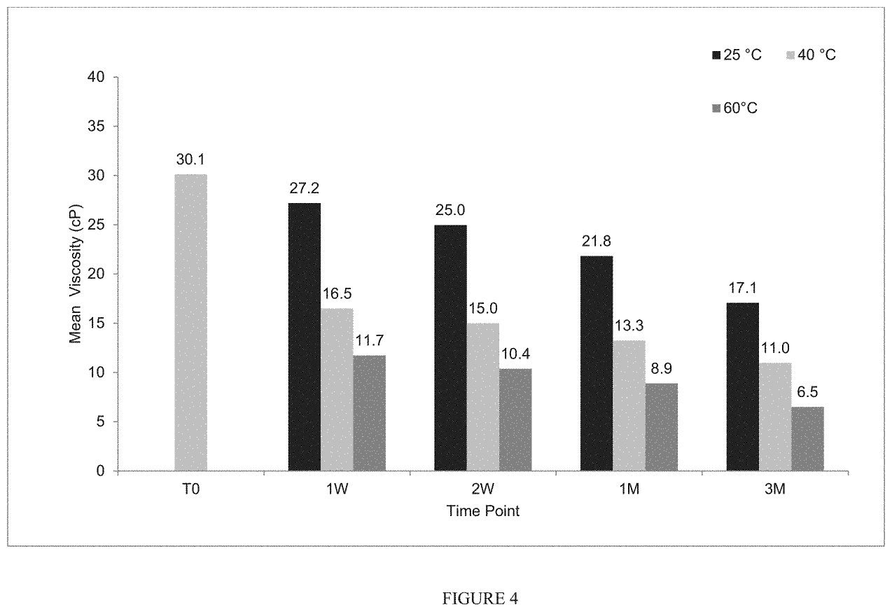

The graph in FIG. 4 shows that the viscosity of 200 mg/mL defibrotide formulations using 20 mM Gly-Gly buffer decreases over time and also decreases with increasing temperature (measured at 25° C., 40° C. and 60° C.). The viscosity decrease as a function of temperature and over time is favorable for drug delivery and product manufacturing, particularly for high concentration products such as HCLFs. The decrease of the viscosity over time, thixotropic behavior, is especially favorable and leads to improved patient convenience and tolerability of these formulations. Defibrotide is a temperature stable product thus the decrease of viscosity at higher temperatures provides additional opportunities for improved patient convenience. For example, if a patient warms up the formulation prior to administration, the viscosity will go down allowing for continued ease of administration particularly for subcutaneous, intradermal, intraocular, inhaled, intraperitoneal and/or intramuscular administration.

### 6.4 Example 4—Osmolality Over Time Using Forced Degradation

It is also important for pharmaceutical products to maintain low osmolality over time and under various conditions. The osmolality of 200 mg/mL defibrotide formulations using Gly-Gly and citrate buffers were therefore measured as a function of both time and temperature using forced degradation studies.

Samples were prepared as described above. Osmolality of the formulations was measured at 25° C., 40° C. and 60° C.

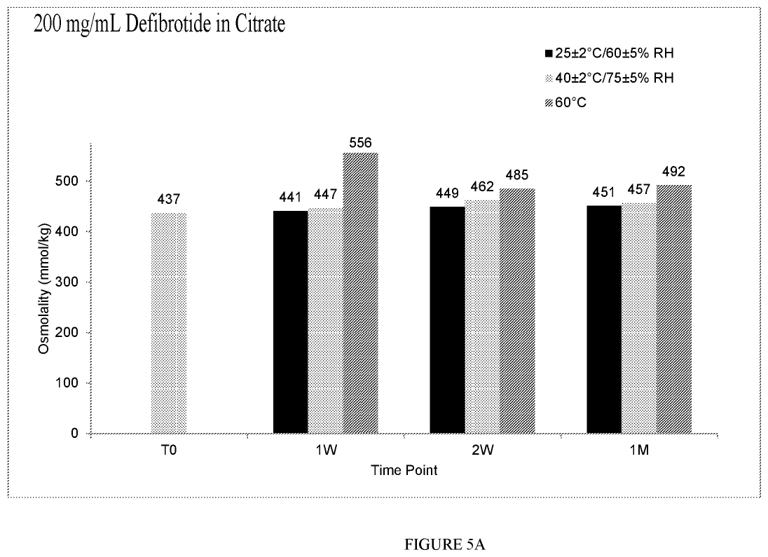

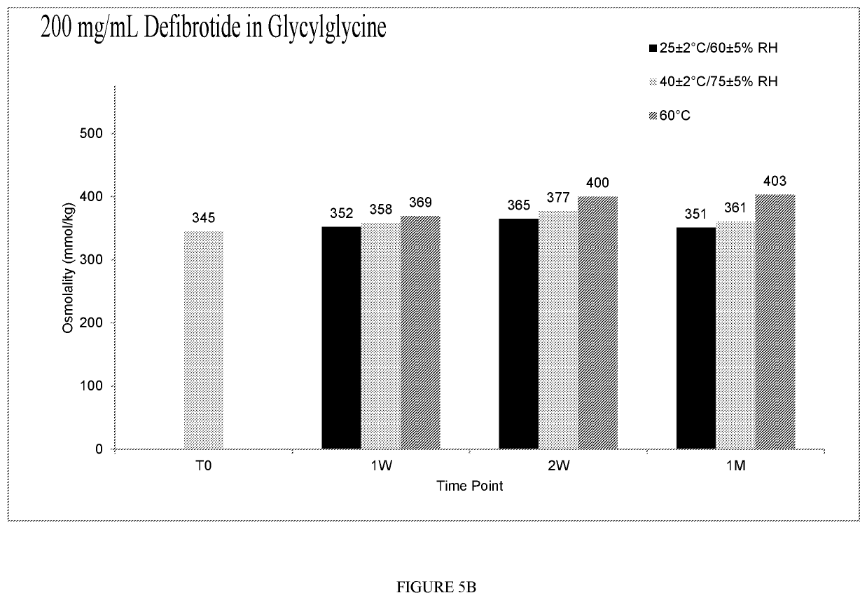

The graph in FIG. 5A shows the osmolality of 200 mg/mL defibrotide formulations using sodium citrate buffer.

The graph in FIG. 5B shows the osmolality of 200 mg/mL defibrotide formulations using Gly-Gly buffer. As seen in these graphs, the osmolality of the Gly-Gly formulations are reduced in comparison to formulations with citrate buffer. Importantly, the osmolality of the Gly-Gly formulations remains consistently low (below about 400 mOsm/kg) over each time point and at every temperature.

### 6.5 Example 5—Physical Stability and Product Profile Under Forced Degradation

The physical stability and product profile of HCLFs of the invention were evaluated using forced degradation studies. Defibrotide within the concentration range of 180 mg/mL and 220 mg/mL formulated in Gly-Gly and/or sodium citrate at pH 3 to pH 10, was evaluated after being stored at 25±2° C./60±5% relative humidity (“RH”), 40±2° C./75±5% RH and 60° C. for up to 3 months. Formulations tested are listed in Table 4 below.

TABLE 4
Summary of Formulations

| For- | Defibrotide | | | |
|---|---|---|---|---|
| mulation | Conc. | | | |
| Code | (mg/mL) | Buffer Type | PH | Surfactant |
| FD1 | 80 | 34 mM Sodium | 7.3 | N/A |
| (Defitelio ® | | Citrate | | |
| Control) | | | | |
| FD2 | 200 | | | |
| FD3 | | 20 mM GlyGly | 7.5 | |
| FD4 | 180 | | | |
| FD5 | 200 | | 3.0 | |
| FD6 | | | 10.0 | |
| FD7 | | 20 mM GlyGly, | 7.3 | |
| | | 10 mM Sodium | | |
| | | Citrate | | |
| FD8 | | 34 mM GlyGly | 7.5 | 0.02% PS-80 |
| FD9 | 220 | 20 mM GlyGly | | N/A |

Based on the appearance, color, clarity, pH and particle count results from the forced degradation stability studies, defibrotide formulated at 200 mg/mL (FD3) in 20 mM Gly-Gly, pH between 7 and 8 was the most stable formulations following intended storage at 25±2° C./60±5% RH and stressed conditions for up to three months.

### 6.6 Example 6—Pharmacokinetics of Nucleic Acid Formulations Using Various Routes of Administration

### 6.6.1 Example 6.1—Intravenous (IV) Infusion, IV Bolus Injection, Subcutaneous (SC) Injection, and Intramuscular (IM) Injection of Defibrotide

The pharmacokinetics (PK) of various defibrotide formulations when administered via a single 2-hr intravenous (IV) infusion, IV bolus injection, subcutaneous (SC) injection, intramuscular (IM) injection, or oral (PO) gavage dose to male Gottingen pigs were compared. In addition, bioavailability of the various extravascular routes of administration was determined relative to IV infusion.

Male Gottingen pigs, or Minipigs, are the industry standard for exploring SC delivery, is an acceptable model for exploring defibrotide SC formulation and delivery options. Each animal received a single administration of defibrotide as listed in the treatment groups in Table 5. Gottingen pigs, n=3 males per group, were assigned to the treatment groups as shown:

TABLE 5
Study Design

| | | Defibrotide | Dose | Nominal Plasma |
|---|---|---|---|---|
| Group | | Dose | Concentration | PK Sampling |
| No. | ROA | (mg/kg) | (mg/mL)^{a} | Time Points |
| 1 | 12-hr IV | 25 | 4 | predose, 0.25, 0.5, 1, 2, 2.083, |
| | infusion | | | 2.25, 2.5, 3, 4, 6, 8, 12, and |
| | | | | 24 hr post-start of infusion |
| 2 | IV bolus | 2.5 | 2.5 | predose, 0.03, 0.083, |
| | | | | 0.25, 0.5, 1, 2, |
| | | | | 3, 4, 5, 6, 8, 12, and 24 hr postdose |
| 3 | SC | 25 | 80 | predose, 0.25, 0.5, |
| 4 | IM | 25 | 80 | 1, 1.5, 2, 3, 4, 5, |
| 5 | PO | 100 | 80 | 6, 8, 10, 12, and |
| | | | | 24 hr postdose |

^{a}The dose volumes were 6.25, 1.0, 0.3125, 0.3125, and 1.25 mL/kg for Groups 1 through 5, respectively

Blood samples were collected and processed to plasma. A Quant-iT OliGreen ssDNA assay kit (Life Technologies) was used to quantify the concentration of defibrotide in pig plasma samples. Briefly, the assay methodology involves aliquoting the sample (in duplicate) into a 96 well plate, the addition of the OliGreen reagent, incubation with stirring (5 min, protected from light), and direct fluorescence measurement (485 excitation, 515 nm cutoff, and 525 nm emission). Assay ranges were 2.5 to 60 μg/mL (high range) and 0.05 to 2.5 μg/mL (low range). The lower limit of quantitation (LLOQ) of the assay was 0.05 μg/mL.

Individual animal defibrotide plasma concentration versus time data were downloaded into WinNonlin Phoenix version 6.3 software (Pharsight, Cary, N.C.) for PK analyses. A noncompartmental IV infusion, IV bolus, or extravascular injection model was used as appropriate to determine the single-dose PK parameters for each animal. Nominal dose and sample collection times (see Table 5) were used in estimating the PK parameters. Background values (range=0.115 to 0.903 μg/mL) were observed at the pre-dose time point in all animals. Therefore, concentration values below 1 μg/mL were treated as <LLOQ and were not used in the analyses. The following parameters were estimated whenever possible:

| | |
|---|---|
| Tmax | Time to maximum observed concentration |
| Cmax | Maximum observed concentration |
| AUC0-t | Area under the concentration-time curve from time = 0 to the time point |
| | with the last measurable concentration, estimated |
| | by the linear trapezoidal rule |
| MRT0-t | Mean residence time from time = 0 to the time point |
| | with the last measurable concentration |
| Cmax/D | Maximum observed concentration divided by dose level |
| AUC0-t/D | Area under the concentration-time curve from time = 0 to the last |
| | measurable concentration, estimated by the linear trapezoidal rule |
| | divided by dose level |
| CL | Calculated for the IV groups as dose divided by AUC0-t |

The bioavailable fraction (F), expressed as a percentage, was calculated for each animal, relative to the IV infusion dose group, as follows based on AUC0-t/D values:

(individual animal SC,IM, or PO AUC0-t/D)/(group mean IV infusion AUC0-t/D)×100%

Defibrotide Plasma Analyses: summarized defibrotide plasma concentrations following IV, SC, IM, or PO dosing to male Gottingen pigs showed the following: after a single IV infusion administration of 25 mg/kg or a single IV bolus administration of 2.5 mg/kg defibrotide, mean plasma concentrations were above 1 μg/mL out to 8 hr post-dose. Following SC or IM administration of 25 mg/kg defibrotide, mean plasma concentrations were greater than 1 μg/mL out to 24 hr post-dose (the last measured time point). Following a 100 mg/kg PO dose, defibrotide plasma concentrations were greater than 1 μg/mL at one time point in one animal (4 hr post-dose in animal 14M) and at three time points in one animal (5, 6, and 12 hr post-dose in animal 13M), but were less than 1 μg/mL at all time points in the third animal (15M).

Pharmacokinetic Analyses: individual animal and summarized PK parameters were also measured and showed the following: after a 2-hr IV infusion of 25 mg/kg defibrotide, the mean Cmax/D was 1.52 (μg/mL)/(mg/kg) and the mean AUC0-t/D value was 3.56 (hr*μg/mL)/(mg/kg). Following IV bolus administration of 2.5 mg/kg defibrotide, the mean Cmax/D was 14.4 (μg/mL)/(mg/kg) and the mean AUC0-t/D value was 8.30 (hr*μg/mL)/(mg/kg).

The Tmax following SC administration of 25 mg/kg defibrotide ranged from 0.25 to 8 hr post-dose, although multiple peaks were observed in the plasma PK profiles. The mean SC bioavailability (% F) was 81.3%. The Tmax following IM administration of 25 mg/kg defibrotide ranged from 0.25 to 0.50 hr post-dose. The mean IM bioavailability was 108%. In contrast, there were very few measurable concentrations following oral administration of 100 mg/kg defibrotide. The mean bioavailability following PO administration was less than 7.2%.

These results show that exposure to defibrotide was prolonged after SC and IM administration, relative to IV administration. The mean MRT last values were 9.26 and 7.36 hr in the SC and IM dose groups, respectively, compared to 1.30 and 2.16 hr in the IV infusion and IV bolus dose groups, respectively.

### 6.6.2 Example 6.3—Outpatient Administration of Defibrotide Formulations

Patient compliance is improved using devices for at home administration. To investigate at home delivery of defibrotide, IV or SC administration options are provided using approved devices for each. FDA approved pumps are used commercially for several products and are suitable for defibrotide delivery. SC administration at home offers the additional benefit of delivering the same dose over 1, 2 or 3 administration times per day rather than 4 as is currently used for Defitelio.

Off-the shelf IV syringe pumps are commonly used for chemotherapy and infectious diseases. These pumps are used to deliver defibrotide formulations which are 80 mg/mL, 150 mg/mL or 200 mg/mL. A Crono Five intrapump (Cane S.p.A.) is a new PCA pump that is appreciated by health care professionals due to its small size and programmable technical features. It is given as a continuous infusion or a bolus or a combination of each. Here, it is used to deliver defibrotide according to Table 6 below:

TABLE 6
Outpatient IV or SC Dosing of Defibrotide

| | DP Volumes | | DP Volumes | | DP Volumes | |
|---|---|---|---|---|---|---|
| | Patient Weight 50 kg | | Patient Weight 80 kg | | Patient Weight 100 kg | |
| Defibrotide | 1,250 mg daily dose | | 2,000 mg daily dose | | 2500 mg daily dose | |
| Concentration | IV | SC | IV | SC | IV | SC |
| 25 mg/kg 1/day | 100% BA | 70% BA | 100% BA | 70% BA | 100% BA | 70% BA |
| 80 mg/mL | 15.6 mL | 22.3 mL | 25.0 mL | 35.7 mL | 31.3 mL | 44.6 mL |
| 150 mg/mL | 8.3 mL | 11.9 mL | 13.3 mL | 19.1 mL | 16.7 mL | 23.8 mL |
| 200 mg/mL | 6.3 mL | 8.9 mL | 10.0 mL | 14.3 mL | 12.5 mL | 17.9 mL |

Off-the shelf syringe pumps are also used for the SC route of administration of defibrotide formulations which are 80 mg/mL, 150 mg/mL or 200 mg/mL. The Cane Crono® (Intrapump) and the Freedom 60® (RMS) are used by patients at home to administer defibrotide according to the table above.

Defitelio® is also administered according to Table 6; however, it is first further diluted as it is normally done for in-hospital IV administration. Defitelio is diluted by a factor of 4 so the concentration is 20 mg/mL. Therefore the above volumes would increase by four times. For example, for a 100 kg patient, they would need to delivery 176 mL; splitting this into 4 treatments per day, the volume of each treatment would be ˜44 mL. This is given over ˜2 hours at a device flow rate of >20 mL/hour, or 1 mL per 3 minutes. Lower Defitelio concentrations are also used. In this case there is more volume per SC dose so a faster flow rate (˜1 mL/minute) is used. These larger volumes can be accommodated in less than 2 hours. In addition, undiluted Defitelio can be administered in shorter and/or fewer treatments (by four times) per day which is a benefit to the patients. Defitelio (undiluted) is administered in two 23 mL doses per day at the 20 mL/hour flow rate.

These improved administration schedules result in less time being connected to an IV infusion per day, less cost burden on the health care system from reduced hospital time and increased quality of life for patients.

### 6.6.3 Example 6.3—Subcutaneous Administration of HCLF Defibrotide Formulations

To further investigate the pharmacokinetics of subcutaneously administered low-viscosity liquid formulations of defibrotide, three different HCLF formulations at 200 mg/mL were compared to a single 2-hr intravenous (IV) infusion or SC injection of Defitelio. In addition, their bioavailability via SC routes of administration was determined relative to IV infusion.

The HCLF formulations at 200 mg/mL were prepared as described above using sodium citrate, Gly-Gly or a combination of these buffers as indicated. Defitelio was administered at 4 mg/mL IV or 80 mg/mL SC using the doses shown in Table 7 below. Male Gottingen pigs (n=3 males per group) received a single administration of the test article listed in Table 7. Defibrotide was analyzed using the analytical method in Example 6.1. The PK parameters were determined similarly as in Example 6.1.

TABLE 7
Administration of Various Formulations

| | Route/Test | Dose | Concentration | Dose Volume |
|---|---|---|---|---|
| Group | Article | (mg/kg) | (mg/mL) | (mL/kg) |
| 1 | 2-hr IV (Defitelio) | 25 | 4 | 6.25 |
| 2 | SC (Defitelio) | 25 | 80 | 0.3125 |
| 3 | SC (HCLF-1) | 25 | 200 | 0.125 |
| 4 | SC (HCLF-2) | 25 | 200 | 0.125 |
| 5 | SC (HCLF-3) | 25 | 200 | 0.125 |

Note:
HCLF1: 34 mM sodium citrate, pH 7.3; HCLF2: 20 mM GlyGly, pH 7.3; HCLF3: 20 mM GlyGly, 10 mM sodium citrate, pH 7.3

The bioavailability expressed as a percent (% F) was calculated as reported above and the results are shown in Table 8 below.

TABLE 8
PK parameters of Defibrotide following SC and IV Administration

| | Cₘₐₓ | Tₘₐₓ | AUC₀₋ₜ | MRT₀₋ₜ | F |
|---|---|---|---|---|---|
| Treatment | μg/mL | h | μg · h/mL | h | % |
| IV, 2-h | 44.8 (5%) | 0.500 | 91.6 (4%) | 2.41 | N/A |
| infusion | | (0.250-1.00) | | (64%) | |
| SC, Defitelio | 7.03 | 0.500 | 56.1 | 9.28 | 61.3% |
| (80 mg/mL) | (17%) | (0.250- | (16%) | (7.6%) | (16%) |
| | | 0.500) | | | |
| SC HCLF1 | 6.40 | 5.00 | 67.2 | 9.91 | 73.4% |
| (200 mg/mL) | (26%) | (4.00-12.0) | (42%) | (3%) | (42%) |
| SC HCLF2 | 7.45 | 1.00 | 59.8 | 10.9 | 65.3% |
| (200 mg/mL) | (62%) | (0.500-12.0) | (30%) | (20%) | (30%) |
| SC HCLF3 | 5.80 | 2.00 | 45.7 | 9.47 | 49.9% |
| (200 mg/mL) | (45%) | (1.00-4.00) | (24%) | (19%) | (24%) |

Mean and % CV values reported except for Tₘₐₓ, for which median and range of observed values (minimum-maximum) are reported.
F (bioavailability): calculated as AUC₀₋ₜ with SC dosing divided by the geometric mean AUC₀₋ₜ for the IV treatment
N/A: not applicable

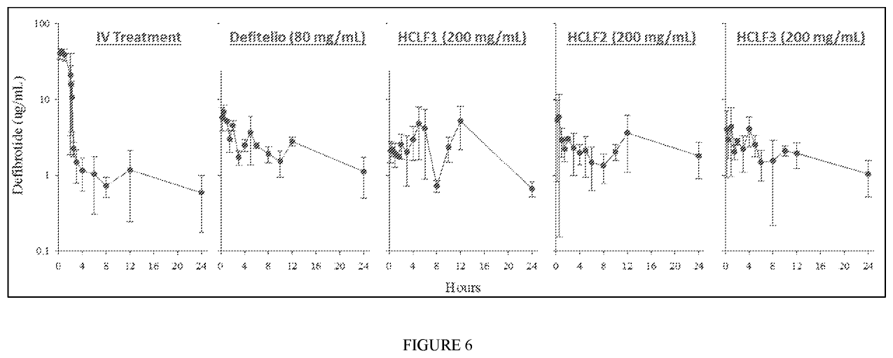

Plasma concentrations of defibrotide and plasma concentration-time data were determined as above and are shown in FIG. 6. The PK profiles in individual minipigs seen in FIG. 6 show multiple absorption peaks for all four SC treatments (Defitelio and the 3 HCLFs). As defibrotide is a mixture of oligonucleotides, the multiple peaks may be due to variation in the rates of absorption of the individual components of defibrotide. Taken together, the results indicate that bioavailability of defibrotide is favorable with SC dosing across all formulations, including the high concentration low-viscosity liquid formulations.

In addition, the mean residence times (MRT) of four SC groups ranged from 9.28-10.9 hours; while MRT of the IV group was just 2.41 hours (Table 8); thus the SC administration provided sustained release of defibrotide at approximately four and half times that of the IV infusion. This is consistent with what was shown in Example 6.1, in that SC administration of defibrotide in low-viscosity, HCLFs prolonged the plasma exposure of defibrotide in comparison to IV administration.

Though not wishing to be bound by any one theory, the extended circulation time by SC route is likely due to the nature of defibrotide HCLFs, which render a sustained release pattern of absorption. The extended circulation of defibrotide by SC administration of HCLFs may present an opportunity to investigate alternate regimens with less frequent dosing and improve quality of life for patients.

### 6.6.4 Example 6.4—Comparison of Pharmacokinetic Profiles of IV and SC Administrations

In an effort to demonstrate the PK comparison of SC HCLF administration, simulations of PK profiles following SC and IV infusion were conducted using the compartmental modeling techniques. A simple 1-compartmental model with or without a first-order absorption process was used to simulate the IV infusion or SC administration PK profile; respectively. In this modeling exercise, mean PK parameters following an IV infusion administration were taken from the package insert of Defitelio®. For PK studies, the clearance and volume of distribution of a drug are typically reported as a function of bioavailability; these are the CL/F and V/F, respectively. For the PK simulation used here, following SC HCLF administration the mean CL/F and V/F were calculated from the IV parameters by assuming a bioavailability of 70%. The absorption rate constant was assumed to be 0.22 h⁻¹, which is similar to that observed in Minipigs. The dose and regimen for the IV infusion were 6.25 mg/kg/infusion (a total daily dose of 25 mg/kg/day), by a 2-hour IV infusion, 4 times a day. The daily dose and regimen for a SC administration was 18 mg/kg/SC administration (a total daily dose of 36 mg/kg/day), 2 times a day. The simulation was conducted for a person with a body-weight of 70 kg. During the simulation, the total AUC following an SC administration was maintained to be the same as that of an IV infusion.

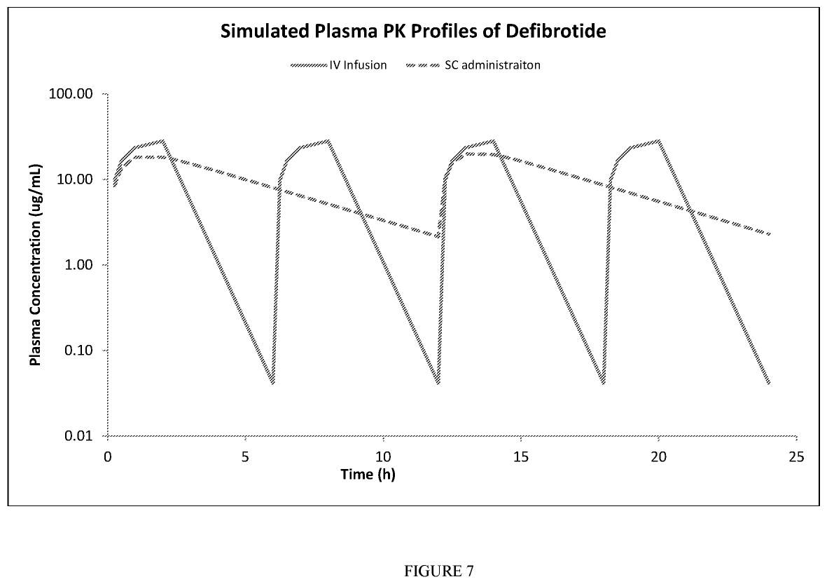

As shown in FIG. 7, the plasma concentration over time profiles demonstrate the slow, constant release of defibrotide following SC administration as opposed to the rapid clearance following each IV infusion. Importantly, the minimum plasma concentration of defibrotide following the SC administration was much higher than that of the IV infusion; while the Cmax of SC administration was similar to that of IV infusion.

The pharmacokinetics of the SC administration represents a profile that allows for continuous plasma exposure of defibrotide which may be important for its pharmacological activity. The peak-to-plasma concentration ratio following SC administration is about 8 as compared to that of the IV infusion which is about 700.

Together, these results demonstrate that subcutaneous administration of defibrotide provides a novel pharmacokinetic profile which differs significantly from the PK of IV infusions, such as those required by currently available defibrotide formulations. Slow and steady release of defibrotide may be critical for its benefit-to-risk profile and the unique subcutaneous pharmacokinetics allow for the development of new doses and/or dosing regimens which may yield better efficacy and improved safety profiles.

### INCORPORATION BY REFERENCE

All patents and publications referenced herein are hereby incorporated by reference in their entireties, including the publications disclosed below.

The publications discussed herein are provided solely for their disclosure prior to the filing date of the present application. Nothing herein is to be construed as an admission that the present disclosure is not entitled to antedate such publication by virtue of prior disclosure.

This application incorporates by reference the following applications in their entireties for all purposes: International Application No. PCT/US2018/045152, filed on Aug. 3, 2018, U.S. application Ser. No. 16/105,319, filed on Aug. 20, 2018, U.S. Provisional Application No. 62/540,657, filed on Aug. 3, 2017, U.S. Provisional Application No. 62/776,500, filed on Dec. 7, 2018 and International Application No. PCT/US2019/064901, filed on Dec. 6, 2019.

1.-86. (canceled)

87. A method for administering a high concentration defibrotide formulation to a patient via a device or a patch,
wherein the device comprises
a. a housing;
b. a reservoir coupled with the housing for containing the high concentration defibrotide formulation;
c. a dispenser in fluid communication with the reservoir and configured for delivery of the high concentration defibrotide formulation into or through a tissue or a cavity of the patient; and
d. a pump, a spring or a plunger configured to actuate delivery of the high concentration defibrotide formulation from the reservoir through the dispenser and into the tissue or the cavity of the patient.

88. The method of claim 87, wherein the high concentration defibrotide formulation comprises between about 50 mg/mL to about 400 mg/mL of defibrotide.

89. The method of claim 87, wherein the high concentration defibrotide formulation comprises 80 mg/mL defibrotide.

90. The method of claim 87, wherein the high concentration defibrotide formulation delivered subcutaneously with the device has a prolonged mean residence time (MRT) compared to a defibrotide formulation delivered intravenously.

91. The method of claim 90, wherein the prolonged MRT ranges from about 7 hours to about 10 hours.

92. The method of claim 87, wherein the high concentration defibrotide formulation delivered subcutaneously with the device exhibits a lower peak-to-trough ratio of plasma concentrations compared to a defibrotide formulation delivered intravenously.

93. The method of claim 87, wherein the high concentration defibrotide formulation further comprises a viscosity reducer at a concentration of between about 5 mM and about 350 mM, and wherein the high concentration defibrotide formulation has a viscosity between about 5 and about 70 cP when measured at between 15° C. and 25° C., and an osmolality between about 240 mOsm/kg and about 1000 mOsm/kg.

94. The method of claim 93, wherein the viscosity reducer is selected from glycylglycine, glycine, a hyaluronidase, sodium citrate, sodium succinate, histidine, TRIS buffer, HEPES buffer, sodium chloride, calcium chloride, magnesium chloride, arginine, lidocaine, benzyl alcohol and/or polysorbate-80, succinic acid, acetic acid, phosphoric acid, tartaric acid, amino acids, cyclodextrin and derivatives, Captsiol®, Polyvinylpyrrolidone (PVP), Kolloidon 12 PF, Kolloidon 17PF (BASF), Kolliphor HS 15 (BASF), MES buffer, Macrogol (15) hydroxystearate, polyethylene glycol (15)-hydroxystearate, polyoxyethylated 12-hydroxystearic acid, or Solutol HS 15.

95. The method of claim 93, wherein the viscosity reducer is glycylglycine at a concentration of between about 20 mM and about 34 mM.

96. The method of claim 87, wherein the high concentration defibrotide formulation further comprises a buffer or excipient so that the nucleic acid is in the form of an alkali metal salt.

97. The method of claim 96, wherein the buffer or excipient is sodium citrate, sodium succinate, histidine, TRIS buffer, HEPES buffer, sodium chloride, arginine, lidocaine, benzyl alcohol and/or polysorbate-80.

98. The method of claim 97, wherein the buffer or excipient is sodium citrate at a concentration of between about 20 mM and about 34 mM.

99. The method of claim 93, wherein the viscosity of the high concentration defibrotide formulation decreases under increasing shear, agitation, temperature, pressure, and/or over time.

100. The method of claim 99, wherein the shear increases during administration.

101. The method of claim 87, wherein the housing is configured to be off-body or removably secured to the patient.

102. The method of claim 87, wherein the device or the patch comprises an adhesive that removably secures the housing of the device or the patch to the patient.

103. The method of claim 87, wherein the reservoir comprises a syringe, a vial, or a cartridge.

104. The method of claim 87, wherein the dispenser is a dispensing needle in fluid communication with the reservoir and configured for delivery of the high concentration defibrotide formulation from the reservoir into or through the tissue or the cavity of the patient.

105. The method of claim 104, wherein the shear increases during administration via the dispensing needle.

106. The method of claim 87, wherein the device further comprises a controller operable to deliver the high concentration defibrotide formulation.

107. The method of claim 87, wherein the high concentration defibrotide formulation is administered subcutaneously, intramuscularly, intradermally, transdermally, nasally, or through or into the peritoneum of the patient.

108. The method of claim 87, wherein the patch is configured for transdermal or intradermal delivery.

109. The method of claim 87, wherein the patch comprises a plurality of dispensing needles, and wherein the high concentration defibrotide formulation is contained within the plurality of dispensing needles.

110. The method of claim 109, wherein the plurality of dispensing needles comprises a coating, and wherein the coating comprises the high concentration defibrotide formulation.

111. The method of claim 87, wherein the high concentration defibrotide formulation is administered at a dosing regimen that provides improved patient quality of life by requiring a reduced administration volume and/or allowing less-frequent administration and/or a shorter duration of administration.

112. The method of claim 87, wherein the high concentration defibrotide formulation is administered in an outpatient or at-home setting.

113. The method of claim 87, wherein the high concentration defibrotide formulation is administered to treat a disease or a condition selected from the group consisting of thrombosis. Hematopoietic Stem Cell Transplantation (HSCT) related complications including sinusoidal obstruction syndrome or hepatic veno-occlusive disease (VOD), Graft versus Host Disease (GvHD), Transplant-Associated Thrombotic Microangiopathy (TA-TMA) or Idiopathic Pneumonia Syndrome, other TMAs including Thrombotic Thrombocytopenic Purpura (TTP) and Hemolytic-Uremic Syndrome (HUS), Acute Myocardial Ischemia, Ischemic Stroke, Ischemia Reperfusion Injury (IRI, including Kidney IRI), treatment and prevention of cytokine release syndrome (CRS) or Chimeric Antigen Receptor (CAR)-T Cell Related Encephalopathy Syndrome (CRES) or CAR-T neurotoxicity, Acute Respiratory Distress Syndrome (ARDS), Sickle Cell Vaso-occlusive Crisis (VOC), Sickle Cell Related Acute Chest Syndrome, Disseminated Intravascular Coagulation (DIC), Sepsis, Renal Insufficiency, other Coronary or Peripheral Artery Diseases, Hematological Malignancies, and Solid Tumors.
